(19) United States
(12) Patent Application Publication
(10) Pub. No.: US 2022/0392638 A1
(43) Pub. Date: Dec. 8, 2022

(54) PRECISION ENRICHMENT OF PATHOLOGY SPECIMENS

(71) Applicant: Foundation Medicine, Inc., Cambridge, MA (US)

(72) Inventors: Jonathan Keith KILLIAN, Cambridge, MA (US); Julia A. ELVIN, Cambridge, MA (US)

(73) Assignee: Foundation Medicine, Inc., Cambridge, MA (US)

(21) Appl. No.: 17/746,712

(22) Filed: May 17, 2022

Related U.S. Application Data
(60) Provisional application No. 63/189,602, filed on May 17, 2021.

(51) Int. Cl. G16H 50/20; C12Q 1/6806; C12Q 1/6886; G16B 15/30; G11B 20/20

(52) U.S. Cl. CPC G16H 50/20; C12Q 1/6806; C12Q 1/6886; G16B 15/30; G11B 20/20; C12Q 1/6869

(57) ABSTRACT

The present disclosure provides methods of improving sequencing analysis and/or extracting nucleic acids, as well as methods, kits, and reagents for detecting nucleic acid molecules extracted from samples in tissues. The disclosure also provides methods for evaluating, identifying, assessing, and/or treating an individual having a cancer.

### CROSS-REFERENCE TO RELATED APPLICATIONS

This application claims priority from U.S. provisional application No. 63/189,602, filed May 17, 2021, the contents of which are incorporated by reference in their entirety.

### FIELD

The present disclosure relates to methods of improving sequencing analysis and methods of extracting nucleic acids, as well as methods of diagnosis, assessment, and treatment of diseases such as cancer.

### BACKGROUND

Cancer represents the phenotypic end-point of multiple genetic lesions that endow cells with a full range of biological properties required for tumorigenesis. Indeed, a hallmark genomic feature of many cancers is the presence of numerous complex chromosome structural aberrations, including translocations, intra-chromosomal inversions, point mutations, deletions, gene copy number changes, gene expression level changes, gene fusions, and germline mutations, among others. The presence of these hallmark genomic features can serve as biomarkers for cancer.

One method of detecting such biomarkers is the analysis of nucleic acids extracted from tumor cells in tissue samples, such as formalin-fixed paraffin-embedded (FFPE) tissues. However, such extractions often fail to enrich for tumor content, or extract an amount of nucleic acids that is insufficient for subsequent analysis such as next-generation sequencing. One method of extracting samples from tissues is laser capture microdissection (LCM). LCM was an attempt to improve the precision and accuracy of specimen enrichments. However, LCM is not scalable. LCM is also wasteful of patient specimen, and has a risk of specimen contamination due to use of water baths as well as other specimen exposures that are unique to LCM.

Accordingly, there exists a need in the art for methods of improving sequencing analysis and extracting nucleic acids, particularly methods that allow for the enrichment of nucleic acids from tumor cells of interest.

All references cited herein, including patents, patent applications and publications, are hereby incorporated by reference in their entirety. To the extent that any reference incorporated by reference conflicts with the instant disclosure, the instant disclosure shall control.

### BRIEF SUMMARY

In some aspects, provided herein is a method of improving sequencing analysis, wherein the method comprises: a) identifying a target region comprising tumor cells of interest in a tissue; b) extracting a sample from the tissue; c) identifying the location of the sample in the tissue; and d) if the location of the sample overlaps with the target region comprising tumor cells of interest, extracting one or more nucleic acids from the sample. In some embodiments, the method further comprises sequencing the one or more nucleic acids from the sample.

In some aspects, provided herein is a method of extracting nucleic acids, wherein the method comprises: a) identifying a target region comprising tumor cells of interest in a tissue; b) extracting a sample from the tissue; c) identifying the location of the sample in the tissue; and d) if the location of the sample overlaps with the target region comprising tumor cells of interest, extracting one or more nucleic acids from the sample. In some embodiments, the method further comprises sequencing the one or more nucleic acids from the sample.

In some embodiments, if the location of the sample does not overlap with the target region comprising tumor cells of interest, steps b) and c) are repeated.

In some embodiments, step b) comprises extracting the sample using a needle. In some embodiments, the needle is punched through the tissue, thereby extracting the sample. In some embodiments, the needle is a disposable needle. In some embodiments, the needle is a 13 gauge needle, 14 gauge needle, a 15 gauge needle, a 16 gauge needle, a 17 gauge needle, a 18 gauge needle, a 19 gauge needle, a 20 gauge needle, or a 21 gauge needle. In some embodiments, the sample extracted from the tissue is about 0.8, 0.9, 1.0, 1.2, 1.4, 1.6, 1.8, 2.0, or 2.3 mm in diameter.

In some embodiments, step b) comprises extracting the sample using laser microdissection (LMD) or a razor blade.

In some embodiments, step c) comprises preparing a slide of a section of the tissue.

In some embodiments, the section of the tissue is stained. In some embodiments, the section of the tissue is Haematoxylin and Eosin (H&E) stained. In some embodiments, step c) is performed by visual inspection. In some embodiments, step c) is performed by a computer system. In some embodiments, step c) is performed using an image analysis system.

In some aspects, provided herein is a method of improving sequencing analysis, wherein the method comprises: a) providing a tissue comprising tumor cells of interest; b) extracting a sample from the tissue; c) assessing the level of enrichment of the tumor cells of interest in the sample and in the remaining tissue; and d) if the level of enrichment of tumor cells of interest in the sample exceeds the level of tumor cells of interest in the remaining tissue, extracting one or more nucleic acids from the sample. In some embodiments, the method further comprises sequencing the one or more nucleic acids from the sample.

In some aspects, provided herein is a method of extracting nucleic acids, wherein the method comprises: a) providing a tissue comprising tumor cells of interest; b) extracting a sample from the tissue; c) assessing the level of enrichment of the tumor cells of interest in the sample and in the remaining tissue; and d) if the level of enrichment of tumor cells of interest in the sample exceeds the level of tumor cells of interest in the remaining tissue, extracting one or more nucleic acids from the sample. In some embodiments, the method further comprises sequencing the one or more nucleic acids from the sample.

In some embodiments, if the level of enrichment of tumor cells of interest in the sample does not exceed the level of tumor cells of interest in the remaining tissue, steps b) and c) are repeated.

In some embodiments, the level of enrichment of tumor cells of interest in the sample is at least 1-fold, 1.1-fold, 1.2-fold, 1.3-fold, 1.4-fold, 1.5-fold, 1.6-fold, 1.7-fold, 1.8-fold, 1.9-fold, or 2-fold higher than the level of tumor cells of interest in the remaining tissue.

In some embodiments, the tissue is from an individual known to have cancer or suspected of having cancer. In some embodiments, the tissue is from a biopsy, optionally a tumor biopsy.

In some embodiments, the tissue is a fixed tissue. In some embodiments, the fixed tissue is selected from the group consisting of a formalin-fixed tissue, an ethanol-fixed tissue, and a methanol-fixed tissue. In some embodiments, the tissue is embedded in an embedding agent. In some embodiments, the embedding agent is resin or paraffin. In some embodiments, the tissue is a formalin-fixed paraffin-embedded (FFPE) tissue. In some embodiments, the tissue is a cryopreserved tissue. In some embodiments, the tissue is a fresh-frozen tissue. In some embodiments, the fresh-frozen tissue is frozen in an optimal cutting temperature (OCT) compound.

In some embodiments, step b) further comprises inspecting the sample, and optionally removing any excess tissue from the sample. In some embodiments, step b) further comprises inspecting the sample, and optionally removing any excess embedding agent from the sample.

In some embodiments, the method further comprises subjecting the one or more nucleic acids extracted from the sample to further processing, optionally wherein the further processing comprises digestion, DnaX treatment, gel electrophoresis, and/or quantification.

In some embodiments, the one or more nucleic acids extracted from the sample comprise RNA and/or DNA.

In some embodiments, the one or more nucleic acids extracted from the sample are analyzed by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping. In some embodiments, the one or more nucleic acids extracted from the sample are analyzed by next-generation sequencing. Methods of next-generation sequencing are described herein.

In some embodiments, the further comprises detecting the presence of one or more biomarkers in the sample. In some embodiments, the method further comprises detecting loss-of-heterozygosity (LOH) of one or more genes of interest in the sample In some embodiments, the method further comprises detecting LOH of a human leukocyte antigen (HLA) gene in the sample. In some embodiments, the method further comprises detecting a loss-of-function mutation in a phosphatase and tensin homolog (PTEN) gene in the sample. In some embodiments, the method further comprises measuring the level of tumor mutational burden (TMB) in the sample. In some embodiments, the method further comprises detecting homozygous single exon loss in the sample.

In some aspects, provided herein is a system comprising: a memory configured to store one or more program instructions; and one or more processors configured to execute the one or more program instructions, wherein the one or more program instructions when executed by the one or more processors are configured to: (a) obtain a plurality of sequence reads of one or more nucleic acids, wherein the one or more nucleic acids are derived from a sample extracted from a tissue according to any one of the methods of the preceding claims; (b) analyze the plurality of sequence reads for the presence of LOH of one or more genes of interest, LOH of a HLA gene, a loss-of-function mutation in a PTEN gene, TMB of at least about 10 mut/Mb or at least about 20 mut/Mb, and/or homozygous single exon loss; and (c) detect, based on the analyzing, LOH of one or more genes of interest, LOH of a HLA gene, a loss-of-function mutation in a PTEN gene, TMB of at least about 10 mut/Mb or at least about 20 mut/Mb, and/or homozygous single exon loss in the sample.

In some aspects, provided herein are methods of identifying an individual having cancer who may benefit from a treatment comprising an anti-cancer therapy, detecting the presence or absence of a cancer in an individual, selecting a therapy for an individual having cancer, identifying one or more treatment options for an individual having cancer, selecting or not selecting a treatment for an individual having cancer, treating or delaying progression of cancer, diagnosing/assessing LOH of one or more genes of interest in a cancer in an individual, diagnosing/assessing LOH of an HLA gene in a cancer in an individual, diagnosing/assessing a loss-of-function mutation in a PTEN gene in a cancer in an individual, diagnosing/assessing the level of TMB in a cancer in an individual, and diagnosing/assessing homozygous single exon loss in a cancer in an individual.

In some embodiments, the incidence of tissue insufficient for analysis is reduced by at least 10%, at least 20%, at least 30%, at least 40% or at least 50% compared to a method not comprising steps c) and/or d). In some embodiments, when the method step b) comprises extracting the sample using a needle and tumor purity is increased at least at least 10%, at least 20%, at least 30%, at least 40% or at least 50% to a method wherein step b) comprises extracting the sample using a razor blade. In some embodiments of any of the above embodiments, the individual is human.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 shows an overview of an exemplary method for performing precision enrichment of pathology specimens from a formalin-fixed paraffin-embedded (FFPE) block.

FIGS. 2A-2B show a representative precision-enriched lung non-small cell lung carcinoma (NSCLC) sample. FIG. 2A shows the post-needle punch slide of the NSCLC sample.

FIG. 2B shows a copy number plot (top panel) and a variant allele frequency (VAF) plot (bottom panel) for a sample subjected to precision enrichment. CN indicates copy number. Together, the data included in the plots show that the tumor purity, which is proportional to all non-CN=2 log ratio, following precision enrichment was 52% and the ploidy for the sample was 2.67 In copy number plot Y-axis shows log ratio of sample genome content and the X-axis shows genomic map position. In the VAF plot, Y-axis shows the VAF and the X-axis shows SNP targets.

FIG. 3 shows the distribution of computed tumor purities for different enrichment methods. The red horizontal line is the 30% tumor purity cut-off for gLOH biomarker detection. The grey horizontal lines show the median for each data set, and the whiskers show the 1.5 interquartile range (IQR). The data included in this plot show improved tumor purity for samples subjected to precision enrichment (NPE and LM-NPE) compared to samples not subjected to precision enrichment (UnE and RBE). UnE indicates an un-enriched sample, RBE indicates razor-blade enriched, NPE and LM-NPE indicate needle-punch enriched, as shown on the x-axis. The y-axis shows the percentage of tumor purity.

FIGS. 4A-4B provide an overview of exemplary comprehensive genomic profiling (CGP) methods.

FIG. 5 shows the relationship between tumor purity, as a result of precision enrichment, and the ability to produce reportable gLOH scores. Tumor purity is significantly higher in the samples with reportable gLOH score (pass) compared to the samples with un-reportable gLOH score (fail). Further, 75% of all samples subjected to precision enrichment had a reportable gLOH scores (pass). The y-axis shows the percentage of tumor purity, “unk” indicates reportable gLOH unknown, “fail” indicates un-reportable gLOH, and “pass” indicates reportable gLOH.

FIG. 6 shows the relationship between tumor purity, as a result of precision enrichment, and the ability to produce reportable CGP scores and companion diagnostic (CDx) scores. Tumor purity is significantly higher in the samples with a reportable CDx score (TRUE) compared to the samples with no reportable CDx score (FALSE). The y-axis shows the percentage of tumor purity, “TRUE” indicates samples with a reportable CDx score, “FALSE indicates samples without a reportable CDx score, and “NULL” indicates samples where it was unknown whether there was a reportable CDx score.

FIG. 7 depicts an exemplary device, “Device 1100,” in accordance with some embodiments.

FIG. 8 depicts an exemplary system, “System 1200,” in accordance with some embodiments.

FIG. 9 depicts a block diagram of an exemplary process for detecting a biomarker (e.g., LOH, HLA LOH, PTEN LOF, TMB, and/or homozygous single exon loss) in a sample extracted from a tissue, in accordance with some embodiments.

FIG. 10 shows the incidence of tumor insufficient for analysis (TIFA) using the present methods compared to control.

### DETAILED DESCRIPTION

The present disclosure is based, at least in part, on the development of methods for extracting nucleic acids from tissues that involves a histologic quality assurance/quality control step to assess the enrichment process. The methods described herein enable testing of specimens that otherwise would produce a level of tissue that was insufficient for analysis of tumor content with prior enrichment methods.

### Definitions

Before describing the invention in detail, it is to be understood that this invention is not limited to particular compositions or biological systems. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “or” is used herein to mean, and is used interchangeably with, the term “and/or”, unless context clearly indicates otherwise.

The terms “about” or “approximately” as used herein refer to the usual error range for the respective value readily known to the skilled person in this technical field, for example, an acceptable degree of error or deviation for the quantity measured given the nature or precision of the measurements. Reference to “about” or “approximately” a value or parameter herein includes (and describes) embodiments that are directed to that value or parameter per se.

As used herein, the term “configured to hybridize to” indicates that a nucleic acid molecule has a nucleotide sequence with sufficient length and sequence complementarity to the nucleotide sequence of a target nucleic acid to allow the nucleic acid molecule to hybridize to the target nucleic acid, e.g., with a Tₘ of at least 65° C. in an aqueous solution of 1×SCC (150 mM sodium chloride and 15 mM trisodium citrate) and 0.1% SDS. Other hybridization conditions may be used when hybridizing a nucleic acid molecule to a target nucleic acid molecule, for example in the context of a described method.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In some embodiments, the individual is human patient, e.g., a human patient having a cancer described herein.

An “effective amount” or a “therapeutically effective amount” of an agent, e.g., an anti-cancer agent, or a pharmaceutical formulation, refers to an amount effective, at dosages and for periods of time necessary, to achieve the desired therapeutic or prophylactic result, e.g., in the treatment or management of a cancer, for example, delaying or minimizing one or more symptoms associated with the cancer. In some embodiments, an effective amount or a therapeutically effective amount of an agent refers to an amount of the agent at dosages and for periods of time necessary, alone or in combination with other therapeutic agents, which provides a therapeutic or prophylactic benefit in the treatment or management of a disease such as a cancer. In some embodiments, an effective amount or a therapeutically effective amount of an agent enhances the therapeutic or prophylactic efficacy of another therapeutic agent or another therapeutic modality.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, delaying progression of disease, alleviation of symptoms, diminishment of any direct or indirect pathological consequences of the disease, decreasing the rate of disease progression, amelioration or palliation of the disease state, and remission or improved prognosis. In some embodiments, the terms “treatment,” “treat,” or “treating” include preventing a disease, such as cancer, e.g., before an individual begins to suffer from a cancer or from re-growth or recurrence of the cancer. In some embodiments, the terms “treatment,” “treat,” or “treating” include inhibiting or reducing the severity of a disease such as a cancer.

“Likely to” or “increased likelihood,” as used herein, refer to an increased probability that an event, item, object, thing or person will occur. Thus, in one example, an individual that is likely to respond to treatment with an anti-cancer therapy, e.g., an anti-cancer therapy provided herein, alone or in combination, has an increased probability of responding to treatment with the anti-cancer therapy alone or in combination, relative to a reference individual or group of individuals. “Unlikely to” refers to a decreased probability that an event, item, object, thing or person will occur relative to a reference individual or group of individuals. Thus, an individual that is unlikely to respond to treatment with an anti-cancer therapy, e.g., an anti-cancer therapy provided herein, alone or in combination, has a decreased probability of responding to treatment with the anti-cancer therapy, alone or in combination, relative to a reference individual or group of individuals.

### I. Methods of Extracting Nucleic Acids

In some aspects, provided herein are methods of extracting nucleic acids. In some embodiments, the method comprises a) identifying a target region comprising tumor cells of interest in a tissue; b) extracting a sample from the tissue; c) identifying the location of the sample in the tissue; and d) if the location of the sample overlaps with the target region comprising tumor cells of interest, extracting one or more nucleic acids from the sample. In some embodiments, if the location of the sample does not overlap with the target region comprising tumor cells of interest, steps b) and c) are repeated. Also provided herein are methods of improving sequencing analysis. In some embodiments, the method comprises a) identifying a target region comprising tumor cells of interest in a tissue; b) extracting a sample from the tissue; c) identifying the location of the sample in the tissue; and d) if the location of the sample overlaps with the target region comprising tumor cells of interest, extracting one or more nucleic acids from the sample. In some embodiments, also provided herein are methods of improving sequencing analysis, wherein the method comprises: a) providing a tissue comprising tumor cells of interest; b) extracting a sample from the tissue; c) assessing the level of enrichment of the tumor cells of interest in the sample and in the remaining tissue; and d) if the level of enrichment of tumor cells of interest in the sample exceeds the level of tumor cells of interest in the remaining tissue, extracting one or more nucleic acids from the sample. In some embodiments, also provided herein are methods of extracting nucleic acids, wherein the method comprises: a) providing a tissue comprising tumor cells of interest; b) extracting a sample from the tissue; c) assessing the level of enrichment of the tumor cells of interest in the sample and in the remaining tissue; and d) if the level of enrichment of tumor cells of interest in the sample exceeds the level of tumor cells of interest in the remaining tissue, extracting one or more nucleic acids from the sample. In some embodiments, if the level of enrichment of tumor cells of interest in the sample does not exceed a threshold level of enrichment, steps b) and c) are repeated. In some embodiments the method further comprises analyzing the one or more nucleic acids from the sample by sequencing, such as next-generation sequencing.

The present disclosure is based, at least in part, on the development of methods for extracting nucleic acids from tissues that comprise tumor cells of interest, such as formalin-fixed paraffin-embedded (FFPE) tissues. The tissues may be from a subject suspected of having cancer, or known to have cancer. Without wishing to be bound by any particular theory, it is believed that the inclusion of a step of analyzing the tissue after extracting the sample is informative with respect to whether the sample has successfully enriched for the tumor cells of interest, or whether a further sample should be extracted from the tissue. For example, a slide of the tissue may be prepared after the sample is extracted in order to determine the degree of overlap with the sample and the tumor cells of interest. Such a histologic quality assurance/quality control step is believed to allow for the assessment of the tumor content enrichment process. Without such a quality assurance/quality control step, samples extracted from tissues have occasionally failed during sequencing analysis due to low tumor purity. These samples were not usable, and were considered to be unusable due to a “likely missed enrichment.” Accordingly, the methods described herein enable testing of specimens that otherwise would produce a level of tissue that was insufficient for analysis of tumor content with prior enrichment methods, and reduce the occurrence of unusable samples due to likely missed enrichments. The successful enrichment of tumor content is of particular importance for the assessment of certain biomarkers that may indicate the presence of cancer and require a relatively high level of tumor content in order to measure the biomarkers. Accordingly, the methods described herein may be used to detect the presence of biomarkers that may have otherwise been undetectable. This is thought to improve the specificity and precision of subsequent sequence analyses of the biomarkers. The methods described herein may be referred to as “precision enrichment” methods, as they involve the precision enrichment of tumor cells of interest, and therefore tumor content, and nucleic acids derived from tumor cells.

In some embodiments, the methods described herein comprise extracting a sample from a tissue using a needle. In some embodiments, the needle is punched through the tissue, thereby extracting the sample. In some embodiments, the needle is a disposable needle. In some embodiments, the needle is a thin-walled needle. In some embodiments, the needle is a blunt tipped needle. In some embodiments, the needle is a stainless steel needle. In some embodiments, the needle is a hypodermic needle. In some embodiments, the needle comprises a Luer-compatible hub. In some embodiments, the needle is a 13 gauge needle, 14 gauge needle, a 15 gauge needle, a 16 gauge needle, a 17 gauge needle, a 18 gauge needle, a 19 gauge needle, a 20 gauge needle, or a 21 gauge needle. In some embodiments, the sample extracted from the tissue is about 0.8, 0.9, 1.0, 1.2, 1.4, 1.6, 1.8, 2.0, or 2.3 mm in diameter. Exemplary methods of using a needle to extract a sample from a tissue are provided in the Examples.

In some embodiments, step b) comprises extracting the sample using laser microdissection (LMD). In some embodiments, step b) comprises extracting the sample using a razor blade.

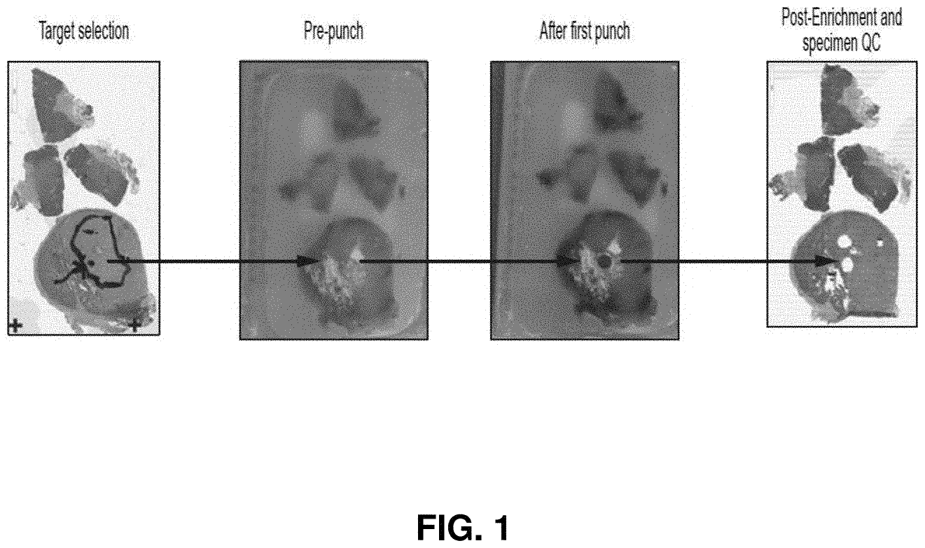

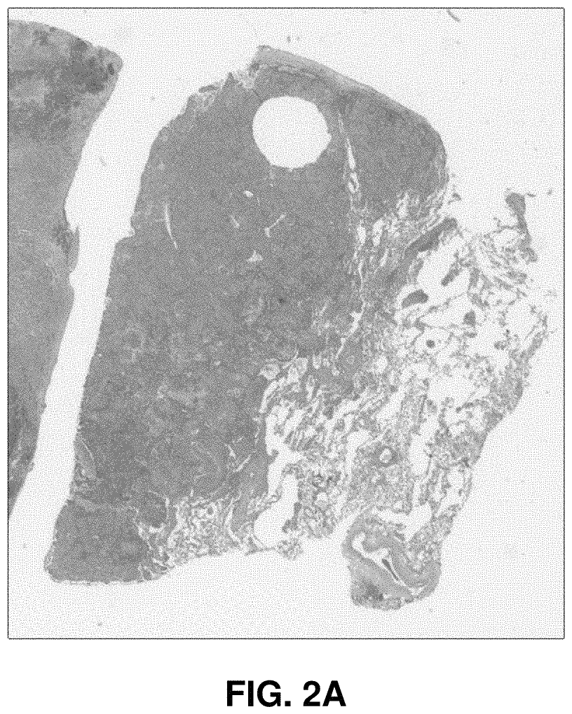

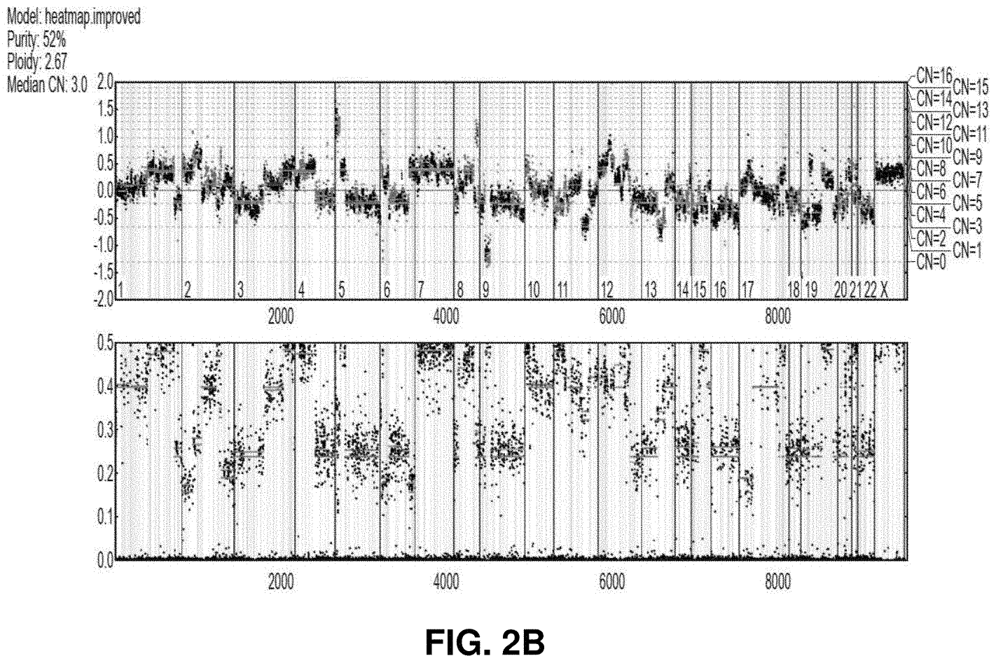

In some embodiments, step c) of the methods described herein comprises preparing a slide of a section of the tissue. In general, the preparation of a slide of a section of the tissue is thought to allow for the assessment of whether the sample (e.g., a sample extracted with a needle as described above), has successfully enriched for the tumor cells of interest. For example, the slide of a section of the tissue may reveal that the position of the sample (e.g., the position of the needle punch where the sample was extracted) overlaps with the position of the tumor cells of interest. Exemplary images of slides of sections of tissues are provided in FIG. 1 and FIG. 2A. As shown in FIG. 1 and FIG. 2A, the position of a needle punch is visible in the section of the tissue. In some embodiments, the section of the tissue is stained. In some embodiments, the section of the tissue is stained in such a way that distinguishes the tumor cells of interest from other cells in the tissue. In some embodiments, the section of the tissue is Haematoxylin and Eosin (H&E) stained. In some embodiments, the section of the tissue is immunostained, e.g., using a detectably-labeled antibody. In some embodiments, the detectably-labeled antibody binds to a protein expressed in the tumor cells of interest. In some embodiments, step c) is performed by visual inspection. For example, in some embodiments, step c) is performed by a pathologist who visually inspects a slide of a section of the tissue and determines whether the location of the sample overlaps with the tumor cells of interest. In some embodiments, step c) is performed by a computer system. For example, in some embodiments, step c) is performed using a computer system that assesses whether the location of the sample overlaps with the tumor cells of interest. In some embodiments, step c) is performed using an image analysis system.

In some embodiments, the level of enrichment of tumor cells of interest in the sample is 95%, 90%, 85%, 80%, 75%, 70%, 65%, 60%, 55%, 50%, or higher. In some embodiments, the level of enrichment of tumor cells of interest in the sample is at least 1-fold, 1.1-fold, 1.2-fold, 1.3-fold, 1.4-fold, 1.5-fold, 1.6-fold, 1.7-fold, 1.8-fold, 1.9-fold, or 2-fold higher than the level of tumor cells of interest in the remaining tissue. In some embodiments, the sample comprises cells comprising least 25%, at least 30%, at least 35%, at least 40%, at least 45%, at least 50%, at least 55%, at least 60%, at least 65%, at least 70%, at least 75%, at least 80%, at least 85%, or at least 90% or more tumor cells of interest.

In some embodiments, the methods provided herein result in decreased incidence of tissue insufficient for analysis (TIFA) compared to other methods. In some embodiments, TIFA is a measure of how often the tumor tissue content is below 20% of a sample. In some embodiments, if the tumor tissue content is too low, the sample is designated TIFA and further analysis, for example, genetic analysis cannot be conducted. In some embodiments, the methods provided herein comprises a) identifying a target region comprising tumor cells of interest in a tissue, b) extracting a sample from the tissue, c) identifying the location of the sample in the tissue, and d) if the location of the sample overlaps with the target region comprising tumor cells of interest, extracting one or more nucleic acids from the sample, wherein the TIFA is reduced compared to a method that does not comprise steps c) and/or d). In some embodiments, TWA is reduced compared to a method that does not comprise step c). In some embodiments, TIFA is reduced compared to a method that does not comprise step d). In some embodiments, the TIFA is reduced compared to a method that does not comprise step c) and step d). In some embodiments, the method comprises extracting the sample from the tissue using a needle, wherein the TIFA is reduced compared to a method comprising extracting the sample using another method (such as a razor blade). In some embodiments, TWA is reduced at least 10%, at least 20%, at least 30%, at least 40% or at least 50% compared to the method not comprising steps c and/or d). In some embodiments TIFA is reduced at least 10%, at least 20%, at least 30%, at least 40% or at least 50% compared to a method not using a needle to extract the sample. In some embodiments, TIFA is reduced 10%-50%, 10%-40%, or 10%-30% using the present methods.

In some embodiments, the methods provided herein result in increased tumor purity (i.e. a higher ratio of tumor cells and/or tumor nucleic acid as compared to non-tumor cells and/or non-tumor nucleic acid). In some embodiments, the methods provided herein comprises a) identifying a target region comprising tumor cells of interest in a tissue, b) extracting a sample from the tissue, c) identifying the location of the sample in the tissue, and d) if the location of the sample overlaps with the target region comprising tumor cells of interest, extracting one or more nucleic acids from the sample, wherein the tumor purity is increased compared to a method that does not comprise steps c) and/or d). In some embodiments, tumor purity is increased compared to a method that does not comprise step c). In some embodiments, the tumor purity is increased compared to a method that does not comprise step d). In some embodiments, the tumor purity is increased compared to a method that does not comprise step c) and step d). In some embodiments, the method comprises extracting the sample from the tissue using a needle, wherein the tumor purity is increased compared to a method comprising extracting the sample using another method (such as a razor blade). In some embodiments, the tumor purity is increased at least 10%, at least 20%, at least 30%, at least 40% or at least 50% compared to the method not comprising steps c and/or d). In some embodiments the tumor purity is increased at least 10%, at least 20%, at least 30%, at least 40% or at least 50% compared to a method not using a needle to extract the sample. In some embodiments, the tumor purity is increased 10%-50%, 10%-40%, or 10%-30% using the present methods.

In some embodiments, the tissue is from an individual known to have cancer. In some embodiments, the tissue is from an individual suspected of having cancer. In some embodiments, the individual is suspected of having any one of the cancers described herein. In some embodiments, the individual is human.

In some embodiments, the tissue is from a biopsy. In some embodiments, a tumor biopsy. In some embodiments, the tissue is a fixed tissue. In some embodiments, the fixed tissue is selected from the group consisting of a formalin-fixed tissue, an ethanol-fixed tissue, and a methanol-fixed tissue. In some embodiments, the tissue is embedded in an embedding agent (e.g., resin or paraffin). In some embodiments, the tissue is an embedded tissue. In some embodiments, the embedded tissue is resin-embedded tissue or a paraffin-embedded tissue. In some embodiments, the tissue is a formalin-fixed paraffin-embedded (FFPE) tissue. In some embodiments, the tissue is a cryopreserved tissue. In some embodiments, the tissue is an archival tissue. In some embodiments, the tissue is a fresh-frozen tissue. In some embodiments, the fresh-frozen tissue is frozen in an optimal cutting temperature (OCT) compound. In some embodiments, the tissue is a primary tissue obtained directly from a source of interest by any appropriate means. For example, in some embodiments, a tissue is obtained by a method chosen from biopsy (e.g., fine needle aspiration or tissue biopsy) and surgery. In one embodiment, the tissue comprises one or more cells associated with a tumor, e.g., tumor cells or tumor-infiltrating lymphocytes (TIL). In one embodiment, the tissue includes one or more premalignant or malignant cells. In one embodiment, the tissue is acquired from a hematologic malignancy (or pre-malignancy), e.g., a hematologic malignancy (or pre-malignancy) described herein. In one embodiment, the tissue is acquired from a cancer, such as a cancer described herein. In some embodiments, the tissue is acquired from a solid tumor, a soft tissue tumor or a metastatic lesion. In other embodiments, the tissue includes tissue or cells from a surgical margin. In some embodiments, the sample comprising tumor cells of interest. In some embodiments, the sample further comprises non-tumor cells. Provided herein are methods comprising extracting a sample from a tissue is from an individual suspected of having cancer. In some embodiments, the tissue comprises tumor cells of interest. In some embodiments, the individual is suspected of having any one of the cancers described herein. In some embodiments, the tumor cells of interest are tumor cells associated with any one of the cancers described herein. In some embodiments, the cancer is acute lymphoblastic leukemia (“ALL”), acute myeloid leukemia (“AML”), adenocarcinoma, adenocarcinoma of the lung, adrenocortical cancer, adrenocortical carcinoma, anal cancer, appendiceal cancer, B-cell derived leukemia, B-cell derived lymphoma, B-cell lymphoma, bladder cancer, brain cancer, breast cancer (e.g., triple negative breast cancer (TNBC) or non-triple negative breast cancer), cancer of the fallopian tube(s), cancer of the testes, carcinoma, cerebral cancer, cervical cancer, cholangiocarcinoma, choriocarcinoma, chronic myelogenous leukemia, central nervous system (CNS) tumor, CNS cancer, colon cancer, colorectal cancer (e.g., colon adenocarcinoma), diffuse intrinsic pontine glioma (DIPG), diffuse large B cell lymphoma (“DLBCL”), embryonal rhabdomyosarcoma (ERMS), endometrial cancer, epithelial cancer, epithelial neoplasm, thymoma, esophageal cancer, Ewing's sarcoma, eye cancer (e.g., uveal melanoma), eyelid cancer, follicular lymphoma (“FL”), gall bladder cancer, gastric cancer, gastrointestinal cancer, glioblastoma, polycythemia vera, glioblastoma multiforme, glioma (e.g., lower grade glioma), gullet cancer, head and neck cancer, a hematological cancer, hepatocellular cancer, hepatocellular carcinoma, Hodgkin's lymphoma (HL), a heavy chain disease, intestinum rectum cancer, renal cancer, kidney cancer (e.g., kidney clear cell cancer, kidney chromophobe cancer, kidney clear cell cancer, kidney papillary cancer), large B-cell lymphoma, large intestine cancer, laryngeal cancer, leucosis, leukemia, liver cancer, lung cancer (e.g., lung adenocarcinoma, or non-small cell lung cancer), lymphoma, mammary gland cancer, melanoma (e.g., metastatic malignant melanoma), Hodgkin's disease, Waldenstrom's macroglobulinemia, Merkel cell carcinoma, mesothelioma, monocytic leukemia, multiple myeloma, myeloma, myogenic sarcoma, nasopharyngeal cancer, neuroblastic-derived CNS tumor (e.g., neuroblastoma (NB)), neuroma, astrocytoma, pilocytic astrocytoma, anaplastic astrocytoma, medulloblastoma, craniopharyngioma, ependymoma, pinealoma, hemangioblastoma, acoustic neuroma, oligodendroglioma, meningioma, vestibular schwannoma, adenoma, metastatic brain tumor, spinal tumor, non-Hodgkin's lymphoma (NHL), oral cancer, oral cavity cancer, osteosarcoma, ovarian cancer, ovarian carcinoma, pancreatic adenocarcinoma, pancreatic cancer, peritoneal cancer, pheochromocytoma, primary mediastinal B-cell lymphoma, primary peritoneal cancer, prostate cancer (e.g., hormone refractory prostate adenocarcinoma), rectal cancer (rectum carcinoma), relapsed or refractory classic Hodgkin's Lymphoma (cHL), salivary gland cancer (e.g., salivary gland tumor), skin cancer, small cell lung cancer, small intestine cancer, soft tissue sarcoma, squamous carcinoma, squamous cell carcinoma (e.g., squamous cell carcinoma of the anogenital region, squamous cell carcinoma of the anus, squamous cell carcinoma of the cervix, squamous cell carcinoma of the esophagus, squamous cell carcinoma of the head and neck (SCHNC), squamous cell carcinoma of the lung, squamous cell carcinoma of the penis, squamous cell carcinoma of the vagina, or squamous cell carcinoma of the vulva), stomach cancer, T-cell derived leukemia, T-cell lymphoma, testicular cancer, testicular tumor, thymic cancer, thyroid cancer (thyroid carcinoma), tongue cancer, tunica conjunctiva cancer, urinary bladder cancer, urothelial cell carcinoma, uterine cancer (e.g., uterine endometrial cancer or uterine sarcoma such as uterine carcinosarcoma), uterine endometrial cancer, uterus cancer, vaginal cancer, vulvar cancer, or Wilms' tumor.

In some embodiments, the cancer is a hematologic cancer (e.g., a hematologic malignancy), such as diffuse large B cell lymphoma (“DLBCL”), Hodgkin's lymphoma (“HL”), Non-Hodgkin's lymphoma (“NHL”), follicular lymphoma (“FL”), acute myeloid leukemia (“AML”), acute lymphoblastic leukemia (“ALL”), multiple myeloma (“MM”), acute lymphoblastic B-cell leukemia, acute lymphoblastic T-cell leukemia, acute myeloblastic leukemia, acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), or hairy cell leukemia. In some embodiments, a hematologic cancer of the disclosure is an acute or a chronic leukemia, such as a lymphoblastic, myelogenous, lymphocytic, or myelocytic leukemia. In some embodiments, a hematologic cancer of the disclosure is a lymphoma (e.g., Hodgkin's lymphoma, such as relapsed or refractory classic Hodgkin's Lymphoma (cHL), a non-Hodgkin's lymphoma, a diffuse large B-cell lymphoma, or a precursor T-lymphoblastic lymphoma), a lymphoepithelial carcinoma, or a malignant histiocytosis.

In some embodiments, the cancer is a solid tumor (e.g., a solid malignancy), such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, osteosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, non-small cell lung cancer (NSCLC), small cell lung carcinoma, bladder carcinoma, lung cancer, epithelial carcinoma, skin cancer, melanoma, neuroblastoma (NB), or retinoblastoma.

In certain embodiments, the cancer is a cancer of the adrenal glands (such as neuroblastoma), bladder cancer (such as urothelial (transitional cell) carcinoma), brain cancer (such as anaplastic astrocytoma or glioblastoma), bone cancer (such as osteosarcoma), bone marrow cancer (such as B-cell acute leukemia (B-ALL) or multiple myeloma), breast cancer (such as invasive ductal carcinoma), head and neck cancer (such as adenocarcinoma, mucoepidermoid carcinoma, squamous cell carcinoma), lymph node cancer, lung cancer (e.g., mucoepidermoid carcinoma, sarcoma, small cell undifferentiated carcinoma, adenocarcinoma, adenosquamous carcinoma, large cell carcinoma, large cell neuroendocrine carcinoma, non-small cell lung carcinoma, non-small cell lung carcinoma not otherwise specified, or squamous cell carcinoma), female reproductive cancer (e.g., cancer of the fallopian tubes such as fallopian tube serous carcinoma; ovarian cancer, such as epithelial carcinoma, epithelial carcinoma not otherwise specified, high grade serous carcinoma, low grade serous carcinoma, serous carcinoma; and uterine cancer, such as carcinosarcoma, endometrial adenocarcinoma, endometrial adenocarcinoma not otherwise specified, papillary serous endometrial adenocarcinoma, leiomyosarcoma, sarcoma, sarcoma not otherwise specified, or smooth muscle tumor of uncertain malignant potential (STUMP)), gallbladder cancer (such as adenocarcinoma), cancer of the gastroesophageal junction (such as adenocarcinoma), lymph node cancer (such as anaplastic large cell lymphoma, B-cell lymphoma, B-cell lymphoma not otherwise specified, diffuse large B cell lymphoma, non-Hodgkin's lymphoma, non-Hodgkin's lymphoma not otherwise specified), colon cancer (such as adenocarcinoma), colorectal cancer, skin cancer (such as melanoma or squamous cell carcinoma), small intestine cancer (adenocarcinoma), soft tissue cancer (such as Ewing sarcoma, fibrosarcoma, histiocytosis, histiocytosis not otherwise specified, juvenile xanthogranuloma or non-Langerhans cell histiocytosis, inflammatory myofibroblastic tumor, leiomyosarcoma, neurofibroma, neuroblastoma, sarcoma not otherwise specified, sarcoma, undifferentiated sarcoma, or an undifferentiated soft tissue cancer), pancreatic cancer (such as carcinoma, carcinoma not otherwise specified, ductal adenocarcinoma, or mucinous cystadenocarcinoma), prostate cancer (such as acinar adenocarcinoma), pericardium cancer (such as mesothelioma), peritoneum cancer (such as mesothelioma), salivary gland cancer (such as carcinoma or carcinoma not otherwise specified), stomach cancer (such as adenocarcinoma, adenocarcinoma not otherwise specified, or diffuse type cancer), kidney cancer (such as renal cell carcinoma or renal cell carcinoma not otherwise specified), thyroid cancer (such as carcinoma, carcinoma not otherwise specified, or papillary carcinoma), or a cancer of unknown primary origin (such as adenocarcinoma, carcinoma, carcinoma not otherwise specified, leiomyosarcoma, malignant neoplasm, malignant neoplasm not otherwise specified, melanoma, myoepithelial carcinoma, squamous cell carcinoma (SCC), or undifferentiated neuroendocrine carcinoma).

In some embodiments, the cancer is a cancer that is recurrent or refractory to one or more prior anti-cancer therapies.

In some embodiments, the cancer is any cancer type provided in Ross et al., Oncologist (2017) 22(12):1444-1450, which is incorporated herein by reference.

In some embodiments, step b) further comprises inspecting the sample, and optionally removing excess tissue from the sample. In some embodiments in which the tissue is embedded in an embedding agent (e.g., paraffin), step b) further comprises inspecting the sample, and optionally removing excess embedding agent from the sample.

In some embodiments, the method further comprises subjecting the one or more nucleic acids extracted from the sample to further processing, optionally wherein the further processing comprises digestion, DnaX treatment, gel electrophoresis, and/or quantification

In some embodiments, the one or more nucleic acids extracted from the sample comprise RNA and/or DNA. In some embodiments, the one or more nucleic acids extracted from the sample comprise genomic DNA, cDNA, or mRNA. In some embodiments, the one or more nucleic acids extracted from the sample comprise RNA and/or DNA from the tumor cells of interest.

II. Analysis of Nucleic Acids Extracted from a Sample Extracted from a Tissue

The one or more nucleic acids extracted from a sample from a tissue, as described above, may be further analyzed, for example, by next-generation sequencing. In general, the methods described herein demonstrate an improved ability to enrich for tumor cells of interest. This allows for the analysis of biomarkers present in the tumor cells that may be useful in detecting cancer. In particular, biomarkers that require a relatively high level of tumor content may be detected with the methods described herein. Exemplary biomarkers that may be detected include loss-of heterozygosity (LOH), LOH of a human leukocyte antigen (HLA) gene (HLA LOH), loss-of-function of a phosphatase and tensin homolog (PTEN) gene (PTEN LOF), tumor mutational burden (TMB), and homozygous single exon loss. Methods of analyzing the nucleic acids extracted from a sample extracted from a tissue are described in detail below. Further, methods of assessing biomarkers are described in detail below.

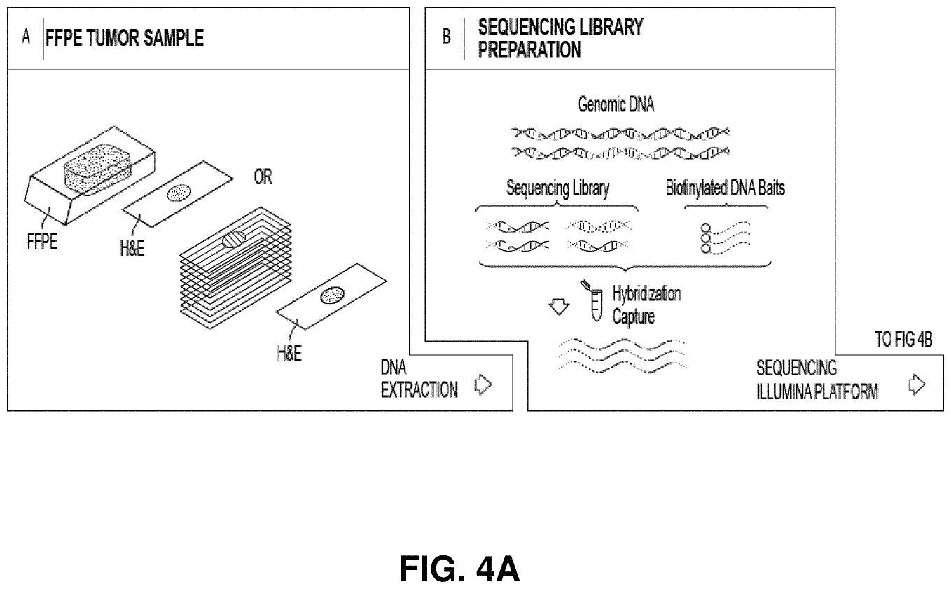

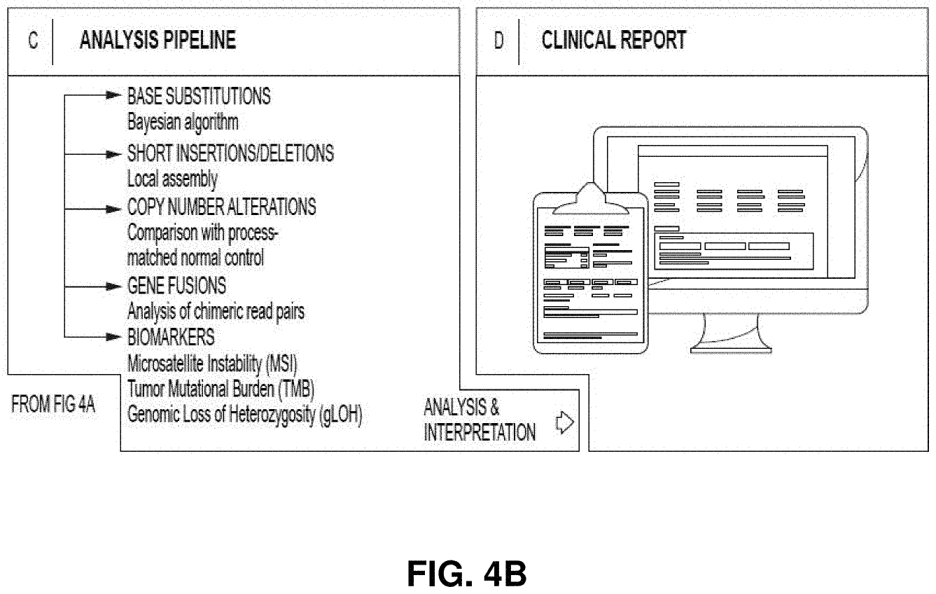

In some embodiments, the one or more nucleic acids extracted from the sample are analyzed by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping. In some embodiments, the one or more nucleic acids extracted from the sample are analyzed by next-generation sequencing. An exemplary method of next-generation sequencing is described in, for example, Frampton, G. M. et al. (2013) Nat. Biotech. 31:1023-1031. In some embodiments, the one or more nucleic acids extracted from the sample are analyzed according to a method as diagrammed in FIGS. 4A-4B.

In some embodiments, the method further comprises: e) optionally, ligating one or more adaptors onto one or more nucleic acids from the one or more nucleic acids extracted from the sample; d) optionally, amplifying nucleic acids from the ligated nucleic acids; f) optionally, capturing a plurality of nucleic acids corresponding to a gene of interest; g) sequencing, by a sequencer, the plurality of nucleic acids to obtain a plurality of sequence reads corresponding to the gene of interest; h) analyzing the plurality of sequence reads; and i) based on the analysis, detecting one or more mutations in a gene of interest. In some embodiments, the plurality of nucleic acids corresponding to the gene of interest is captured from the amplified nucleic acids by hybridization with a bait molecule.

In some embodiments, the method further comprises detecting the presence of one or more biomarkers (e.g., LOH, HLA LOH, PTEN LOF, TMB, and/or homozygous single exon loss) in the sample extracted from the tissue, as described in further detail below. In general, since the methods of the present disclosure result in an enrichment in the level of tumor cells of interest in a sample, they allow for the detection of biomarkers that may be difficult to detect or undetectable using other methods of extracting nucleic acids from tissues.

In some embodiments, a biomarker (e.g., LOH, HLA LOH, PTEN LOF, TMB, and/or homozygous single exon loss) nucleic acid molecule of the disclosure is detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety.

In Situ Hybridization Methods

In some embodiments, a biomarker (e.g., LOH, HLA LOH, PTEN LOF, TMB, and/or homozygous single exon loss) nucleic acid molecule of the disclosure is detected in the sample extracted from the tissue using an in situ hybridization method, such as a fluorescence in situ hybridization (FISH) method.

In some embodiments, FISH analysis is used to identify the chromosomal rearrangement resulting in the mutations as described herein. In some embodiments, FISH analysis is used to identify an RNA molecule comprising a biomarker nucleic acid described herein. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH. In some embodiments, “break-away FISH” is used in the methods provided herein. In break-away FISH, at least one probe targeting a fusion junction or breakpoint and at least one probe targeting an individual gene of the fusion, e.g., at one or more exons and or introns of the gene, are utilized. In normal cells (i.e., cells not having a fusion nucleic acid molecule described herein), both probes are observed (or a secondary color is observed due to the close proximity of the two genes of the gene fusion); and in cells having a fusion nucleic acid molecule described herein, only a single gene probe is observed due to the presence of a rearrangement resulting in the fusion nucleic acid molecule.

Array-Based Methods

In some embodiments, a biomarker (e.g., LOH, HLA LOH, PTEN LOF, TMB, and/or homozygous single exon loss) nucleic acid molecule of the disclosure is detected in the sample extracted from the tissue using an array-based method, such as array-based comparative genomic hybridization (CGH) methods. In array-based CGH methods, a first sample of nucleic acids (e.g., from a sample, such as from a tumor) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor) is labeled and hybridized to a second array (with identical content) and absolute signals are read. Copy number differences are calculated based on absolute signals from the two arrays.

Amplification-Based Methods

In some embodiments, a biomarker (e.g., LOH, HLA LOH, PTEN LOF, TMB, and/or homozygous single exon loss) nucleic acid molecule of the disclosure is detected in the sample extracted from the tissue using an amplification-based method. As is known in the art, in such amplification-based methods, a sample of nucleic acids, such as a sample obtained from an individual or from a tumor, is used as a template in an amplification reaction (e.g., Polymerase Chain Reaction (PCR)) using one or more oligonucleotides or primers, e.g., such as one or more oligonucleotides or primers provided herein. The presence of a biomarker nucleic acid molecule of the disclosure in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

Sequencing

In some embodiments, a biomarker (e.g., LOH, HLA LOH, PTEN LOF, TMB, and/or homozygous single exon loss) nucleic acid molecule of the disclosure is detected in the sample extracted from the tissue using a sequencing method. Any method of sequencing known in the art can be used to detect a biomarker nucleic acid molecule provided herein. Exemplary sequencing methods that may be used to detect a biomarker nucleic acid molecule provided herein include those based on techniques developed by Maxam and Gilbert or Sanger. Automated sequencing procedures may also be used, e.g., including sequencing by mass spectrometry.

In some embodiments, a biomarker (e.g., LOH, HLA LOH, PTEN LOF, TMB, and/or homozygous single exon loss) nucleic acid molecule of the disclosure is detected in the sample extracted from the tissue using hybrid capture-based sequencing (hybrid capture-based NGS), e.g., using adaptor ligation-based libraries. See, e.g., Frampton, G. M. et al. (2013) Nat. Biotech. 31:1023-1031. In some embodiments, a biomarker nucleic acid molecule of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10⁵ molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect a biomarker nucleic acid molecule provided herein include, without limitation, the HeliScope Gene Sequencing system from Helicos BioSciences (Cambridge, Mass., USA), the PacBio RS system from Pacific Biosciences (Menlo Park, Calif., USA), massively parallel short-read sequencing such as the Solexa sequencer and other methods and platforms from Illumina Inc. (San Diego, Calif., USA), 454 sequencing from 454 LifeSciences (Branford, Conn., USA), Ion Torrent sequencing from ThermoFisher (Waltham, Mass., USA), or the SOLiD sequencer from Applied Biosystems (Foster City, Calif., USA). Additional exemplary methods and platforms that may be used to detect a biomarker nucleic acid molecule provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, Calif., USA).

In some embodiments, the one or more nucleic acids extracted from the sample are analyzed by next-generation sequencing. In some embodiments, the method further comprises: e) optionally, ligating one or more adaptors onto one or more nucleic acids from the one or more nucleic acids extracted from the sample; d) optionally, amplifying nucleic acids from the ligated nucleic acids; f) optionally, capturing a plurality of nucleic acids corresponding to one or more genes of interest; g) sequencing, by a sequencer, the plurality of nucleic acids to obtain a plurality of sequence reads corresponding to the one or more genes of interest; h) analyzing the plurality of sequence reads; and i) based on the analysis, detecting one or more mutations in a gene of interest. In some embodiments, prior to step e) the one or more nucleic acids extracted from the sample are fragmented, optionally wherein the one or more nucleic acids extracted from the sample are fragmented by sonication. In some embodiments, the fragmented one or more nucleic acids extracted from the sample are end-repaired. In some embodiments, the end-repaired, fragmented one or more nucleic acids extracted from the sample are dA-tailed or dT-tailed. In some embodiments, the one or more nucleic acids extracted from the sample are prepared for sequencing according to the method described in Frampton, G. M. et al. (2013) Nat. Biotech. 31:1023-1031.

Biomarker Detection

In some aspects, provided herein are reagents for detecting a biomarker (e.g., LOH, HLA LOH, PTEN LOF, TMB, and/or homozygous single exon loss) nucleic acid molecule of the disclosure or a fragment thereof, e.g., in the nucleic acids extracted from the samples, as described herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid, e.g., a nucleic acid that comprises a biomarker nucleic acid molecule described herein or a fragment or portion thereof. Provided herein are baits suitable for the detection of a biomarker nucleic acid molecule of the disclosure. In some embodiments, the bait comprises a capture nucleic acid molecule configured to hybridize to a target nucleic acid molecule comprising a biomarker nucleic acid molecule provided herein, or a fragment or portion thereof. In some embodiments, the capture nucleic acid molecule is configured to hybridize to the biomarker nucleic acid molecule of the target nucleic acid molecule. Also provided herein are probes, e.g., nucleic acid molecules, suitable for the detection of a biomarker nucleic acid molecule provided herein. In some embodiments, a probe provided herein comprises a nucleic acid sequence configured to hybridize to a target nucleic acid molecule comprising a biomarker nucleic acid molecule provided herein, or a fragment or portion thereof. In some embodiments, the probe comprises a nucleic acid sequence configured to hybridize to the biomarker nucleic acid molecule, or the fragment or portion thereof, of the target nucleic acid molecule. In some embodiments, the probe comprises a nucleic acid sequence configured to hybridize to a fragment or portion of the biomarker nucleic acid molecule of the target nucleic acid molecule. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

### Loss-of-Heterozygosity (LOH) of One or More Genes of Interest, e.g., a Human Leukocyte Antigen (HLA) Gene

In some embodiments, provided herein are methods that comprise detecting loss-of-heterozygosity (LOH) of one or more genes of interest in a sample extracted from a tissue, as described herein. In some embodiments, provided herein are methods that comprise detecting LOH of a human leukocyte antigen (HLA) gene in a sample extracted from a tissue, as described herein. Exemplary methods of detecting LOH of a HLA gene are described in International Application No. PCT/US2021/019982, which is hereby incorporated by reference in its entirety.

In other embodiments, the gene of interest is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEGS, NNAT, GNAS, or GATA5.

In some embodiments, any one of the methods described above further comprises detecting loss-of-heterozygosity (LOH) of a human leukocyte antigen (HLA) gene in a sample extracted from a tissue as described herein. In some embodiments according to any of the embodiments described herein, the HLA gene encodes a major histocompatibility (MHC) class I molecule. In some embodiments, the methods further comprise, after determining the adjusted allele frequency: determining that the gene has undergone loss-of-heterozygosity (LOH) based at least in part on the adjusted allele frequency.

In yet some other aspects, provided herein are methods for detecting loss-of-heterozygosity (LOH) of a human leukocyte antigen (HLA) gene in a sample extracted from a tissue as described herein. In some embodiments, the methods comprise: a) obtaining an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) applying an objective function to measure a difference between the relative binding propensity and the observed allele frequency of the HLA allele; d) applying an optimization model to minimize the objective function; e) determining an adjusted allele frequency of the HLA allele based on the optimization model and the observed allele frequency; and f) determining that LOH has occurred when the adjusted allele frequency of the HLA allele is less than a predetermined threshold. In some embodiments, the HLA gene is a human HLA-A, HLA-B, or HLA-C gene. In some embodiments, the plurality of sequence reads was obtained by sequencing nucleic acids obtained from the sample. In some embodiments, the methods are for detecting loss-of-heterozygosity (LOH) of a polymorphic gene of interest in the sample. In some embodiments, the methods comprise: a) obtaining an observed allele frequency for an allele of a gene of interest, wherein observed allele frequency corresponds to frequency of nucleic acid(s) encoding at least a portion of the allele as detected among a plurality of sequence reads corresponding to the gene, wherein the plurality of sequence reads was obtained by sequencing nucleic acids encoding the gene or a portion thereof as captured by hybridization with a bait molecule; b) obtaining a relative binding propensity for the allele to the bait molecule, wherein the relative binding propensity of the allele corresponds to propensity of nucleic acid encoding at least a portion of the allele to bind the bait molecule in the presence of nucleic acids encoding portions of one or more other alleles; c) applying an objective function to measure a difference between the relative binding propensity and the observed allele frequency of the allele; d) applying an optimization model to minimize the objective function; e) determining an adjusted allele frequency of the allele based on the optimization model and the observed allele frequency; and f) determining that LOH has occurred when the adjusted allele frequency of the allele is less than a predetermined threshold. In some embodiments, the polymorphic gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEGS, NNAT, GNAS, or GATA5.

In yet some other aspects, any of the methods of the present disclosure further comprise measuring TMB, e.g., in a sample extracted from a tissue as described herein. In some embodiments, the methods comprise determining LOH and assessing TMB, e.g., in a sample of the present disclosure. As demonstrated herein, HLA LOH and high TMB (and optionally intact HLA gene(s)) may be predictive of increased overall survival, increased probability of greater survival, and/or increased likelihood of response to ICI therapy, e.g., as compared to HLA LOH without high TMB. In some embodiments, high TMB refers to a TMB of greater than or equal to 10 mutations/Mb or greater than or equal to 13 mutations/Mb. In some embodiments, TMB is obtained from a plurality of sequence reads, e.g., a plurality of sequence reads obtained by sequencing nucleic acids at least a portion of a genome (such as from an enriched or unenriched sample). In some embodiments, TMB is determined based on a number of non-driver somatic coding mutations per megabase of genome sequenced.

In some embodiments, any of the methods of the present disclosure comprise acquiring knowledge of LOH of the HLA gene (e.g., in a sample extracted from a tissue) and acquiring knowledge of TMB (e.g., in a sample extracted from a tissue). In some embodiments, any of the methods of the present disclosure comprise detecting LOH of the HLA gene (e.g., in a sample extracted from a tissue) and acquiring knowledge of TMB (e.g., in a sample extracted from a tissue). In some embodiments, any of the methods of the present disclosure comprise acquiring knowledge of LOH of the HLA gene (e.g., in a sample extracted from a tissue) and detecting or determining TMB (e.g., in a sample extracted from a tissue). In some embodiments, any of the methods of the present disclosure comprise detecting LOH of the HLA gene (e.g., in a sample extracted from a tissue) and detecting or determining TMB (e.g., in a sample extracted from a tissue). In some embodiments, the samples used to detect/determine LOH and TMB are the same. In some embodiments, the samples used to detect/determine LOH and TMB are different.

### Phosphatase and Tensin Homolog (PTEN)

Phosphatase and tensin homolog (PTEN) deleted on chromosome 10 is one of the most frequently disrupted tumor suppressors in cancer. The lipid phosphatase activity of PTEN antagonizes the phosphatidylinositol 3-kinase (PI3K)/AKT/mTOR pathway to repress tumor cell growth and survival. Accordingly, a loss-of-function mutation in a PTEN gene can serve as a biomarker for cancer.

In some embodiments, provided herein are methods that comprise detecting a loss-of-function mutation in a phosphatase and tensin homolog (PTEN) gene in the sample (e.g., a sample extracted from a tissue as described herein). In some embodiments, the loss-of-function mutation in the PTEN gene comprises one or more of an insertion, deletion or substitution of one or more nucleotides, a genomic rearrangement, an alteration in a promoter, a gene fusion, or a copy number alteration.

### Tumor Mutational Burden (TMB)

In some embodiments, provided herein are methods that comprise measuring the level of tumor mutational burden (TMB) in the sample (e.g., a sample extracted from a tissue as described herein). In some embodiments, the methods provided herein comprise acquiring knowledge that a sample extracted from a tissue has a tumor mutational burden of at least about 10 mut/Mb or at least about 20 mut/Mb. In some embodiments, acquiring knowledge that the sample extracted from a tissue has a tumor mutational burden of at least about 10 mut/Mb or at least about 20 mut/Mb comprises measuring the level of tumor mutational burden in a sample, e.g., in a sample extracted from a tissue obtained from an individual. In some embodiments, the methods provided herein comprise detecting a tumor mutational burden of at least about 10 mut/Mb or at least about 20 mut/Mb in a sample extracted from a tissue. In some embodiments, the methods comprise administering an effective amount of an immunotherapy responsive to knowledge that the sample extracted from a tissue has a tumor mutational burden of at least about 10 mut/Mb or at least about 20 mut/Mb. In some embodiments, the methods comprise providing a report to a party.

In some embodiments, tumor mutational burden is assessed in sample from an individual, such as sample extracted from a tissue as described herein. In some embodiments, the sample from the individual comprises a tumor biopsy. In some embodiments, the sample from the individual comprises nucleic acids.

In some embodiments, tumor mutational burden is measured using any suitable method known in the art. For example, tumor mutational burden may be measured using whole-exome sequencing (WES), next-generation sequencing, whole genome sequencing, gene-targeted sequencing, or sequencing of a panel of genes, e.g., panels including cancer-related genes. See, e.g., Melendez et al., Transl Lung Cancer Res (2018) 7(6):661-667. In some embodiments, tumor mutational burden is measured using gene-targeted sequencing, e.g., using a nucleic acid hybridization-capture method, e.g., coupled with sequencing. See, e.g., Fancello et al., J Immunother Cancer (2019) 7:183.

In some embodiments, tumor mutational burden is measured according to the methods provided in WO2017151524A1, which is hereby incorporated by reference in its entirety.

In some embodiments, tumor mutational burden is measured in the sample extracted from the tissue by whole exome sequencing. In some embodiments, tumor mutational burden is measured in the sample using next-generation sequencing. In some embodiments, tumor mutational burden is measured in the sample using whole genome sequencing. In some embodiments, tumor mutational burden is measured in the sample by gene-targeted sequencing. In some embodiments, tumor mutational burden is measured on between about 0.8 Mb and about 1.1 Mb of sequenced DNA. In some embodiments, tumor mutational burden is measured on any of about 0.8 Mb, about 0.81 Mb, about 0.82 Mb, about 0.83 Mb, about 0.84 Mb, about 0.85 Mb, about 0.86 Mb, about 0.87 Mb, about 0.88 Mb, about 0.89 Mb, about 0.9 Mb, about 0.91 Mb, about 0.92 Mb, about 0.93 Mb, about 0.94 Mb, about 0.95 Mb, about 0.96 Mb, about 0.97 Mb, about 0.98 Mb, about 0.99 Mb, about 1 Mb, about 1.01 Mb, about 1.02 Mb, about 1.03 Mb, about 1.04 Mb, about 1.05 Mb, about 1.06 Mb, about 1.07 Mb, about 1.08 Mb, about 1.09 Mb, or about 1.1 Mb of sequenced DNA. In some embodiments, tumor mutational burden is measured on about 0.8 Mb of sequenced DNA.

In some embodiments, the sample extracted from the tissue has a high tumor mutational burden, e.g., of at least about 10 mut/Mb. In some embodiments, the sample extracted from the tissue has a tumor mutational burden of at least about 10 mut/Mb. In some embodiments, the sample extracted from the tissue has a tumor mutational burden of at least about 20 mut/Mb. In some embodiments, the sample extracted from the tissue has a tumor mutational burden of any of between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, the sample extracted from a tissue has a tumor mutational burden of any of between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb.

In some embodiments, measuring tumor mutational burden comprises assessing mutations in a sample extracted from the tissue derived from a cancer in an individual. In some embodiments, measuring tumor mutational burden comprises assessing mutations in a sample extracted from the tissue derived from a cancer in an individual, and in a matched normal sample, e.g., a sample from the individual derived from a tissue or other source that is free of the cancer.

### Homozygous Single Exon Loss

In general, homozygous single exon loss refers to the deletion of both copies of a given exon. In some embodiments, provided herein are methods that comprise detecting homozygous single exon loss in the sample (e.g., a sample extracted from a tissue as described herein). In some embodiments, the homozygous single exon loss is detected in the one or more nucleic acids from the sample by whole exome sequencing, whole genome sequencing, or gene-targeted sequencing

### Systems, Software, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform the method according to any of the embodiments described herein.

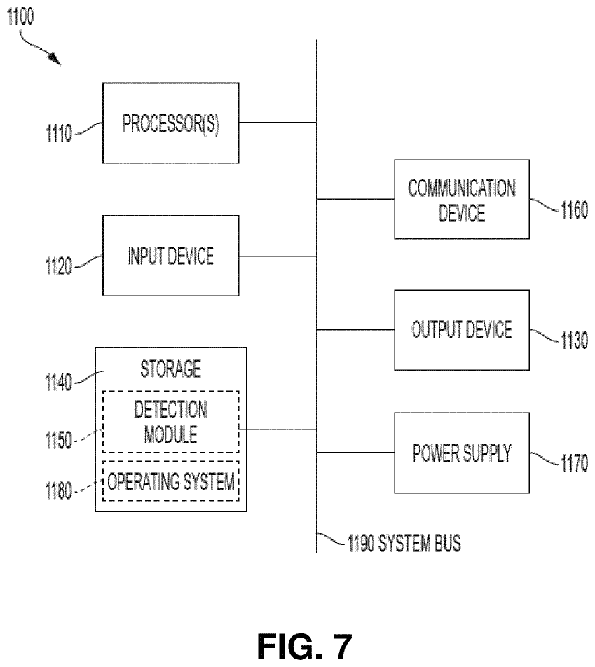

FIG. 7 illustrates an example of a computing device in accordance with one embodiment. Device 1100 can be a host computer connected to a network. Device 1100 can be a client computer or a server. As shown in FIG. 7, device 1100 can be any suitable type of microprocessor-based device, such as a personal computer, workstation, server or handheld computing device (portable electronic device) such as a phone or tablet. The device can include, for example, one or more of processor(s) 1110, input device 1120, output device 1130, storage 1140, communication device 1160, power supply 1170, operating system 1180, and system bus 1190. Input device 1120 and output device 1130 can generally correspond to those described herein, and can either be connectable or integrated with the computer.

Input device 1120 can be any suitable device that provides input, such as a touch screen, keyboard or keypad, mouse, or voice-recognition device. Output device 1130 can be any suitable device that provides output, such as a touch screen, haptics device, or speaker.

Storage 1140 can be any suitable device that provides storage (e.g., an electrical, magnetic or optical memory including a RAM (volatile and non-volatile), cache, hard drive, or removable storage disk). Communication device 1160 can include any suitable device capable of transmitting and receiving signals over a network, such as a network interface chip or device. The components of the computer can be connected in any suitable manner, such as via a wired media (e.g., a physical bus, ethernet, or any other wire transfer technology) or wirelessly (e.g., Bluetooth®, Wi-Fi®, or any other wireless technology). For example, in FIG. 7, the components are connected by System Bus 1190.

Detection module 1150, which can be stored as executable instructions in storage 1140 and executed by processor(s) 1110, can include, for example, the processes that embody the functionality of the present disclosure (e.g., as embodied in the devices as described herein).

Detection module 1150 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1140, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Detection module 1150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

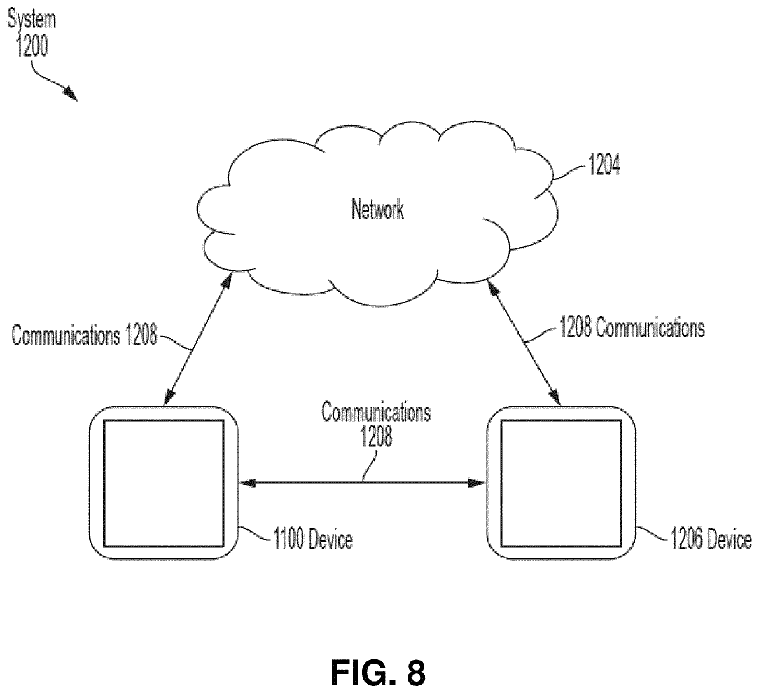

Device 1100 may be connected to a network (e.g., Network 1204, as shown in FIG. 8 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1100 can implement any operating system (e.g., Operating System 1180) suitable for operating on the network. Detection module 1150 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, Operating System 1180 is executed by one or more processors, e.g., Processor(s) 1110.

Device 1100 can further include Power Supply 1170, which can be any suitable power supply.

FIG. 8 illustrates an example of a computing system in accordance with one embodiment. In System 1200, Device 1100 (e.g., as described above and illustrated in FIG. 7) is connected to Network 1204, which is also connected to Device 1206. In some embodiments, Device 1206 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system. Devices 1100 and 1206 may communicate, e.g., using suitable communication interfaces via Network 1204, such as a Local Area Network (LAN), Virtual Private Network (VPN), or the Internet. In some embodiments, Network 1204 can be, for example, the Internet, an intranet, a virtual private network, a cloud network, a wired network, or a wireless network. Devices 1100 and 1206 may communicate, in part or in whole, via wireless or hardwired communications, such as Ethernet, IEEE 802.11b wireless, or the like. Additionally, Devices 1100 and 1206 may communicate, e.g., using suitable communication interfaces, via a second network, such as a mobile/cellular network. Communication between Devices 1100 and 1206 may further include or communicate with various servers such as a mail server, mobile server, media server, telephone server, and the like. In some embodiments, Devices 1100 and 1206 can communicate directly (instead of, or in addition to, communicating via Network 1204), e.g., via wireless or hardwired communications, such as Ethernet, IEEE 802.11b wireless, or the like. In some embodiments, Devices 1100 and 1206 communicate via Communications 1208, which can be a direct connection or can occur via a network (e.g., Network 1204).

One or all of Devices 1100 and 1206 generally include logic (e.g., http web server logic) or is programmed to format data, accessed from local or remote databases or other sources of data and content, for providing and/or receiving information via Network 1204 according to various examples described herein.

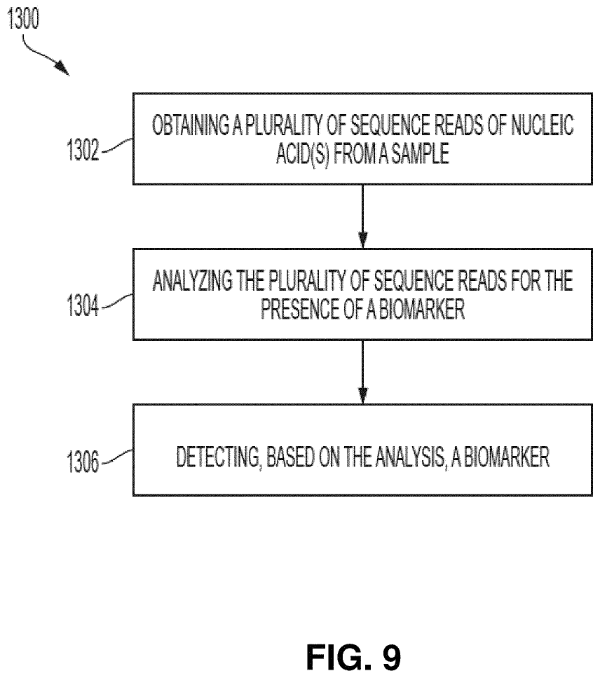

FIG. 9 illustrates an exemplary process 1300 for detecting a biomarker (e.g., LOH, HLA LOH, PTEN LOF, TMB, and/or homozygous single exon loss) in a sample extracted from a tissue, in accordance with some embodiments. Process 1300 is performed, for example, using one or more electronic devices implementing a software program. In some examples, process 1300 is performed using a client-server system, and the blocks of process 1300 are divided up in any manner between the server and a client device. In other examples, the blocks of process 1300 are divided up between the server and multiple client devices. Thus, while portions of process 1300 are described herein as being performed by particular devices of a client-server system, it will be appreciated that process 1300 is not so limited. In some embodiments, the executed steps can be executed across many systems, e.g., in a cloud environment. In other examples, process 1300 is performed using only a client device or only multiple client devices. In process 1300, some blocks are, optionally, combined, the order of some blocks is, optionally, changed, and some blocks are, optionally, omitted. In some examples, additional steps may be performed in combination with the process 1300. Accordingly, the operations as illustrated (and described in greater detail below) are exemplary by nature and, as such, should not be viewed as limiting.

At block 1302, a plurality of sequence reads of one or more nucleic acids is obtained, wherein the one or more nucleic acids are derived from a sample obtained from an individual. In some embodiments, the sample is obtained from an individual having a cancer, such as a cancer described herein. In some embodiments, the sequence reads are obtained using a sequencer, e.g., as described herein or otherwise known in the art. In some embodiments, the nucleic acid(s) comprise one or more nucleic acids corresponding to a biomarker (e.g., LOH, HLA LOH, PTEN LOF, TMB, and/or homozygous single exon loss) of the present disclosure, or portion thereof. Optionally, prior to obtaining the sequence reads, the sample is purified, enriched (e.g., for nucleic acid(s) corresponding to a biomarker gene of the present disclosure, or portion thereof), and/or subjected to PCR amplification. At block 1304, an exemplary system (e.g., one or more electronic devices) analyzes the plurality of sequence reads for the presence of one or more mutations in a biomarker, or a portion thereof. At block 1306, the system detects (e.g., based on the analysis) one or more mutations in a biomarker, or a portion thereof, in the sample.

### III. Diagnostic and Therapeutic Methods and Kits

Methods of Diagnosing, Assessing, Screening, Monitoring or Predicting

In some aspects, provided herein are methods of diagnosing or assessing a biomarker (e.g., LOH, HLA LOH, PTEN LOF, TMB, and/or homozygous single exon loss) in a cancer, such as a cancer provided herein, in an individual. In some embodiments, the methods comprise acquiring knowledge of the presence of a biomarker (e.g., LOH, HLA LOH, PTEN LOF, TMB, and/or homozygous single exon loss) nucleic acid molecule provided herein in a sample extracted from a tissue obtained from the individual. In some embodiments, the methods comprise detecting a biomarker (e.g., LOH, HLA LOH, PTEN LOF, TMB, and/or homozygous single exon loss) nucleic acid molecule provided herein in a sample extracted from a tissue obtained from the individual. In some embodiments, the biomarker (e.g., LOH, HLA LOH, PTEN LOF, TMB, and/or homozygous single exon loss) nucleic acid molecule is detected in a sample extracted from a tissue obtained from the individual using any method known in the art, such as one or more of the methods of detection of biomarker (e.g., LOH, HLA LOH, PTEN LOF, TMB, and/or homozygous single exon loss) nucleic acid molecules described herein. In some embodiments, the methods further comprise providing a diagnosis or an assessment of the biomarker (e.g., LOH, HLA LOH, PTEN LOF, TMB, and/or homozygous single exon loss) nucleic acid molecule. In some embodiments, the diagnosis or assessment identifies the presence or absence of the biomarker (e.g., LOH, HLA LOH, PTEN LOF, TMB, and/or homozygous single exon loss) nucleic acid molecule in the sample. In some embodiments, the diagnosis or assessment identifies the cancer, such as a cancer provided herein, as likely to respond to an anti-cancer therapy, e.g., an anti-cancer therapy provided herein. In some embodiments, the presence of the biomarker (e.g., LOH, HLA LOH, PTEN LOF, TMB, and/or homozygous single exon loss) nucleic acid molecule in the sample identifies the cancer as likely to respond to an anti-cancer therapy, e.g., an anti-cancer therapy provided herein. In some embodiments, the sample is a sample described herein. In some embodiments, the sample extracted from the tissue comprises cells from the cancer or is obtained from cells from the cancer. In some embodiments, the individual has a cancer, is suspected of having a cancer, is being tested for a cancer, is being treated for a cancer, or is being tested for a susceptibility to a cancer, e.g., a cancer described herein.

In some aspects, provided herein are methods of diagnosing or assessing a cancer in an individual, e.g., a cancer provided herein. In some embodiments, the methods of diagnosing or assessing cancer comprise detecting a biomarker (e.g., LOH, HLA LOH, PTEN LOF, TMB, and/or homozygous single exon loss) nucleic acid molecule provided herein in a sample extracted from a tissue obtained from the individual, e.g., a sample comprising cells from the cancer. In some embodiments, the methods comprise detecting a biomarker (e.g., LOH, HLA LOH, PTEN LOF, TMB, and/or homozygous single exon loss) nucleic acid molecule described herein in a sample extracted from a tissue obtained from the individual using any method known in the art, such as one or more of the methods of detection of biomarker (e.g., LOH, HLA LOH, PTEN LOF, TMB, and/or homozygous single exon loss) nucleic acid molecules described herein. In some embodiments, detection of a biomarker (e.g., LOH, HLA LOH, PTEN LOF, TMB, and/or homozygous single exon loss) nucleic acid molecule described herein, or a fragment thereof, in a sample obtained from the individual identifies the cancer as likely to respond to an anti-cancer therapy, e.g., an anti-cancer therapy provided herein. In some embodiments, the presence of a biomarker (e.g., LOH, HLA LOH, PTEN LOF, TMB, and/or homozygous single exon loss) nucleic acid molecule described herein, or a fragment thereof, in a sample extracted from a tissue obtained from the individual identifies the cancer as likely to respond to an anti-cancer therapy, e.g., an anti-cancer therapy provided herein. In some embodiments, the methods further comprise providing a diagnosis or an assessment of the cancer or of the fusion nucleic acid molecule. In some embodiments, the diagnosis or assessment identifies the cancer as likely to respond to an anti-cancer therapy, e.g., an anti-cancer therapy provided herein. In some embodiments, the diagnosis or assessment identifies the presence or absence of the biomarker (e.g., LOH, HLA LOH, PTEN LOF, TMB, and/or homozygous single exon loss) nucleic acid molecule in the sample extracted from the tissue.

In some aspects, provided herein are methods of predicting survival of an individual having a cancer, e.g., a cancer provided herein. In some embodiments, the individual is being treated with an anti-cancer therapy, such as an anti-cancer therapy described herein. In some embodiments, the methods comprise acquiring knowledge of a biomarker (e.g., LOH, HLA LOH, PTEN LOF, TMB, and/or homozygous single exon loss) nucleic acid molecule provided herein in a sample extracted from a tissue from the individual. In some embodiments, the methods comprise detecting a biomarker (e.g., LOH, HLA LOH, PTEN LOF, TMB, and/or homozygous single exon loss) nucleic acid molecule provided herein in a sample extracted from a tissue from the individual. In some embodiments, responsive to acquiring knowledge of a biomarker (e.g., LOH, HLA LOH, PTEN LOF, TMB, and/or homozygous single exon loss) nucleic acid molecule provided herein in the sample, the individual is predicted to have longer survival after treatment with an anti-cancer therapy, e.g., an anti-cancer therapy provided herein, for example, as compared to an individual whose cancer does not exhibit the biomarker (e.g., LOH, HLA LOH, PTEN LOF, TMB, and/or homozygous single exon loss) nucleic acid molecule. In some embodiments, responsive to detecting a biomarker (e.g., LOH, HLA LOH, PTEN LOF, TMB, and/or homozygous single exon loss) nucleic acid molecule provided herein in the sample, the individual is predicted to have longer survival after treatment with an anti-cancer therapy, e.g., an anti-cancer therapy provided herein, for example, as compared to an individual whose cancer does not exhibit the biomarker (e.g., LOH, HLA LOH, PTEN LOF, TMB, and/or homozygous single exon loss) nucleic acid molecule. In some embodiments, the methods further comprise providing a diagnosis or an assessment. In some embodiments, the diagnosis or assessment identifies the presence or absence of the biomarker (e.g., LOH, HLA LOH, PTEN LOF, TMB, and/or homozygous single exon loss) nucleic acid molecule in the sample. In some embodiments, the diagnosis or assessment identifies the individual as being predicted to have longer survival after treatment with an anti-cancer therapy, e.g., an anti-cancer therapy provided herein, for example, as compared to an individual whose cancer does not exhibit the biomarker (e.g., LOH, HLA LOH, PTEN LOF, TMB, and/or homozygous single exon loss) nucleic acid molecule. In some embodiments, the sample is a sample extracted from a tissue as described herein. In some embodiments, the sample comprises cells from the cancer.

In some aspects, provided herein are methods of screening an individual having cancer, suspected of having cancer, being tested for cancer, being treated for cancer, or being tested for a susceptibility to cancer, e.g., a cancer provided herein. In some embodiments, the individual is being treated with an anti-cancer therapy, such as an anti-cancer therapy described herein. In some embodiments, the methods comprise acquiring knowledge of a biomarker (e.g., LOH, HLA LOH, PTEN LOF, TMB, and/or homozygous single exon loss) nucleic acid molecule provided herein in a sample extracted from a tissue from the individual. In some embodiments, the methods comprise detecting a biomarker (e.g., LOH, HLA LOH, PTEN LOF, TMB, and/or homozygous single exon loss) nucleic acid molecule provided herein in a sample extracted from a tissue from the individual. In some embodiments, responsive to acquiring knowledge of a biomarker (e.g., LOH, HLA LOH, PTEN LOF, TMB, and/or homozygous single exon loss) nucleic acid molecule provided herein in the sample, the individual is predicted to have increased risk of cancer recurrence, aggressive cancer, anti-cancer therapy resistance, or poor prognosis, for example, as compared to an individual whose cancer does not exhibit the biomarker (e.g., LOH, HLA LOH, PTEN LOF, TMB, and/or homozygous single exon loss) nucleic acid molecule. In some embodiments, responsive to detecting a biomarker (e.g., LOH, HLA LOH, PTEN LOF, TMB, and/or homozygous single exon loss) nucleic acid molecule provided herein in the sample extracted from a tissue, the individual is predicted to have increased risk of cancer recurrence, aggressive cancer, anti-cancer therapy resistance, or poor prognosis, for example, as compared to an individual whose cancer does not exhibit the biomarker (e.g., LOH, HLA LOH, PTEN LOF, TMB, and/or homozygous single exon loss) nucleic acid molecule. In some embodiments, the methods further comprise providing a diagnosis or an assessment. In some embodiments, the diagnosis or assessment identifies the presence or absence of the biomarker (e.g., LOH, HLA LOH, PTEN LOF, TMB, and/or homozygous single exon loss) nucleic acid molecule in the sample extracted from a tissue. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the biomarker (e.g., LOH, HLA LOH, PTEN LOF, TMB, and/or homozygous single exon loss) nucleic acid molecule. In some embodiments, the sample is a sample extracted from a tissue as described herein. In some embodiments, the sample extracted from a tissue comprises cells from the cancer.

In some embodiments, the methods further comprise selectively enriching for one or more nucleic acids comprising biomarker (e.g., LOH, HLA LOH, PTEN LOF, TMB, and/or homozygous single exon loss) nucleotide sequences to produce an enriched sample, e.g., using a reagent known in the art or provided herein, such as a bait, probe, or oligonucleotide described herein.

Anti-Cancer Therapies Cancers

Certain aspects of the present disclosure relate to anti-cancer therapies, as well as methods for identifying an individual who may benefit from treatment with an anti-cancer therapy, methods for selecting an anti-cancer therapy for treating an individual, methods for identifying an anti-cancer therapy as a treatment option, methods for treating or delaying progression of cancer comprising administration of an anti-cancer therapy, uses for anti-cancer therapies (e.g., in methods of treating or delaying progression of cancer in an individual, or in methods for manufacturing a medicament for treating or delaying progression of cancer), and the like. These methods and uses are based, at least in part, on the detection of biomarkers (e.g., LOH, HLA LOH, PTEN LOF, TMB, and/or homozygous single exon loss) from tumor cells of interest as extracted from samples from tissues, as described above. Without wishing to be bound to theory, it is thought that the methods described herein allow for the detection of biomarkers (e.g., LOH, HLA LOH, PTEN LOF, TMB, and/or homozygous single exon loss) that require a relatively high level of tumor content in order to be detectable, and that therefore may not be detectable with other methods of extracting samples. Without wishing to be bound to theory, it is thought that these biomarkers can identify patients that would benefit from appropriate anti-cancer therapies such as one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In some embodiments, the anti-cancer therapy comprises a cyclin-dependent kinase (CDK) inhibitor. In some embodiments, the CDK inhibitor inhibits CDK4. In some embodiments, the CDK inhibitor inhibits Cyclin D/CDK4. In some embodiments, the anti-cancer therapy/CDK inhibitor is (a) a small molecule that inhibits one or more enzymatic activities of CDK4, (b) an antibody that inhibits one or more activities of CDK4 (e.g., by binding to and inhibiting one or more activities of CDK4, binding to and inhibiting expression of CDK4, and/or binding to and inhibiting one or more activities of a cell expressing CDK4, such as by inducing antibody-dependent cellular cytotoxicity, ADCC, or phagocytosis, ADCP), or (c) a nucleic acid that inhibits expression of CDK4 (e.g., an antisense oligonucleotide, miRNA, siRNA, morpholino, CRISPR-based therapeutic, and the like). In some embodiments, the CDK inhibitor inhibits CDK4 and CDK6. In some embodiments, the CDK inhibitor is a small molecule inhibitor of CDK4 (e.g., a competitive or non-competitive inhibitor). Non-limiting examples of CDK inhibitors include palbociclib, ribociclib, and abemaciclib, as well as pharmaceutically acceptable salts thereof.

In some embodiments, the anti-cancer therapy comprises a murine double minute 2 homolog (MDM2) inhibitor. In some embodiments, the anti-cancer therapy/MDM2 inhibitor is (a) a small molecule that inhibits one or more activities of MDM2 (e.g., binding to p53), (b) an antibody that inhibits one or more activities of MDM2 (e.g., by binding to and inhibiting one or more activities of MDM2, binding to and inhibiting expression of MDM2, and/or binding to and inhibiting one or more activities of a cell expressing MDM2, such as by inducing antibody-dependent cellular cytotoxicity, ADCC, or phagocytosis, ADCP), or (c) a nucleic acid that inhibits expression of MDM2 (e.g., an antisense oligonucleotide, miRNA, siRNA, morpholino, CRISPR-based therapeutic, and the like). In some embodiments, the MDM2 inhibitor is a small molecule inhibitor of MDM2 (e.g., a competitive or non-competitive inhibitor). Non-limiting examples of MDM2 inhibitors include nutlin-3a, RG7112, idasanutlin (RG7388), AMG-232, MI-63, MI-291, MI-391, MI-77301 (SAR405838), APG-115, DS-3032b, NVP-CGM097, and HDM-201 (siremadlin), as well as pharmaceutically acceptable salts thereof. In some embodiments, the MDM2 inhibitor inhibits or disrupts interaction between MDM2 and p53.

In some embodiments, the anti-cancer therapy comprises one or more of an antimetabolite, DNA-damaging agent, or platinum-containing therapeutic (e.g., 5-azacitadine, 5-fluorouracil, acadesine, busulfan, carboplatin, cisplatin, chlorambucil, CPT-11, cytarabine, daunorubicin, decitabine, doxorubicin, etoposide, fludarabine, gemcitabine, idarubicin, radiation, oxaliplatin, temozolomide, topotecan, trabectedin, GSK2830371, or rucaparib); a pro-apoptotic agent (e.g., a BCL2 inhibitor or downregulator, SMAC mimetic, or TRAIL agonist such as ABT-263, ABT-737, oridonin, venetoclax, combination of venetoclax and an anti-CD20 antibody such as obinutuzumab or rituximab, 1396-11, ABT-10, SM-164, D269H/E195R, or rhTRAIL); a tyrosine kinase inhibitor (e.g., as described herein); an inhibitor of RAS, RAF, MEK, or the MAPK pathway (e.g., AZD6244, dabrafenib, LGX818, PD0325901, pimasertib, trametinib, or vemurafenib); an inhibitor of PI3K, mTOR, or Akt (e.g., as described herein); a CDK inhibitor (e.g., as described herein); a PKC inhibitor (e.g., LXS196 or sotrastaurin); an antibody-based therapeutic (e.g., an anti-PD-1 or anti-PDL1 antibody such as atezolizumab, pembrolizumab, nivolumab, or spartalizumab; an anti-CD20 antibody such as obinutuzumab or rituximab; or an anti-DR5 antibody such as drozitumab); a proteasome inhibitor (e.g., bortezomib, carfilzomib, ixazomib, or MG-132); an HDAC inhibitor (e.g., SAHA or VPA); an antibiotic (e.g., actinomycin D); a zinc-containing therapeutic (e.g., zinc or ZMC1); an HSP inhibitor (e.g., geldanamycin); an ATPase inhibitor (e.g., archazolid); a mitotic inhibitor (e.g., paclitaxel or vincristine); metformin; methotrexate; tanshinone IIA; and/or P5091.

In some embodiments, the anti-cancer therapy comprises a tyrosine kinase inhibitor. In some embodiments, the anti-cancer therapy/tyrosine kinase inhibitor is (a) a small molecule that inhibits one or more enzymatic activities of a tyrosine kinase, (b) an antibody that inhibits one or more activities of a tyrosine kinase (e.g., by binding to and inhibiting one or more activities of the tyrosine kinase, binding to and inhibiting expression, such as cell surface expression, of the tyrosine kinase, and/or binding to and inhibiting one or more activities of a cell expressing the tyrosine kinase, such as by inducing antibody-dependent cellular cytotoxicity, ADCC, or phagocytosis, ADCP), or (c) a nucleic acid that inhibits expression of a tyrosine kinase (e.g., an antisense oligonucleotide, miRNA, siRNA, morpholino, CRISPR-based therapeutic, and the like). In some embodiments, the tyrosine kinase inhibitor is a small molecule inhibitor of a tyrosine kinase (e.g., a competitive or non-competitive inhibitor). Non-limiting examples of tyrosine kinase inhibitors include imatinib, crenolanib, linifanib, ninetedanib, axitinib, dasatinib, imetelstat, midostaurin, pazopanib, sorafenib, sunitinb, motesanib, masitinib, vatalanib, cabozanitinib, tivozanib, OSI-930, Ki8751, telatinib, dovitinib, tyrphostin AG 1296, and amuvatinib, as well as pharmaceutically acceptable salts thereof.

In some embodiments, the anti-cancer therapy comprises a mitogen-activated protein kinase (MEK) inhibitor. In some embodiments, the MEK inhibitor inhibits one or more activities of MEK1 and/or MEK2. In some embodiments, the anti-cancer therapy/MEK inhibitor is (a) a small molecule that inhibits one or more enzymatic activities of MEK, (b) an antibody that inhibits one or more activities of MEK (e.g., by binding to and inhibiting one or more activities of MEK, binding to and inhibiting expression of MEK, and/or binding to and inhibiting one or more activities of a cell expressing MEK, such as by inducing antibody-dependent cellular cytotoxicity, ADCC, or phagocytosis, ADCP), or (c) a nucleic acid that inhibits expression of MEK (e.g., an antisense oligonucleotide, miRNA, siRNA, morpholino, CRISPR-based therapeutic, and the like). In some embodiments, the MEK inhibitor is a small molecule inhibitor of MEK (e.g., a competitive or non-competitive inhibitor). Non-limiting examples of MEK inhibitors include trametinib, cobimetinib, binimetinib, CI-1040, PD0325901, selumetinib, AZD8330, TAK-733, GDC-0623, refametinib, pimasertib, RO4987655, RO5126766, WX-544, and HL-085, as well as pharmaceutically acceptable salts thereof. In some embodiments, the anti-cancer therapy inhibits one or more activities of the Raf/MEK/ERK pathway, including inhibitors of Raf, MEK, and/or ERK.

In some embodiments, the anti-cancer therapy comprises a mammalian target of rapamycin (mTOR) inhibitor. In some embodiments, the anti-cancer therapy/mTOR inhibitor is (a) a small molecule that inhibits one or more enzymatic activities of mTOR, (b) an antibody that inhibits one or more activities of mTOR (e.g., by binding to and inhibiting one or more activities of mTOR, binding to and inhibiting expression of mTOR, and/or binding to and inhibiting one or more activities of a cell expressing mTOR, such as by inducing antibody-dependent cellular cytotoxicity, ADCC, or phagocytosis, ADCP), or (c) a nucleic acid that inhibits expression of mTOR (e.g., an antisense oligonucleotide, miRNA, siRNA, morpholino, CRISPR-based therapeutic, and the like). In some embodiments, the mTOR inhibitor is a small molecule inhibitor of mTOR (e.g., a competitive inhibitor, such as an ATP-competitive inhibitor, or a non-competitive inhibitor, such as a rapamycin analog). Non-limiting examples of mTOR inhibitors include temsirolimus, everolimus, ridaforolimus, dactolisib, GSK2126458, XL765, AZD8055, AZD2014, MLN128, PP242, NVP-BEZ235, LY3023414, PQR309, PKI587, and OSI027, as well as pharmaceutically acceptable salts thereof. In some embodiments, the anti-cancer therapy inhibits one or more activities of the Akt/mTOR pathway, including inhibitors of Akt and/or mTOR.

In some embodiments, the anti-cancer therapy comprises a PI3K inhibitor or Akt inhibitor. In some embodiments, the PI3K inhibitor inhibits one or more activities of PI3K. In some embodiments, the anti-cancer therapy/PI3K inhibitor is (a) a small molecule that inhibits one or more enzymatic activities of PI3K, (b) an antibody that inhibits one or more activities of PI3K (e.g., by binding to and inhibiting one or more activities of PI3K, binding to and inhibiting expression of PI3K, and/or binding to and inhibiting one or more activities of a cell expressing PI3K, such as by inducing antibody-dependent cellular cytotoxicity, ADCC, or phagocytosis, ADCP), or (c) a nucleic acid that inhibits expression of PI3K (e.g., an antisense oligonucleotide, miRNA, siRNA, morpholino, CRISPR-based therapeutic, and the like). In some embodiments, the PI3K inhibitor is a small molecule inhibitor of PI3K (e.g., a competitive or non-competitive inhibitor). Non-limiting examples of PI3K inhibitors include GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDC0032, IPI145, INK1117 (MLN1117), SAR260301, KIN-193 (AZD6482), duvelisib, GS-9820, GSK2636771, GDC-0980, AMG319, pazobanib, and alpelisib (BYL719, Piqray), as well as pharmaceutically acceptable salts thereof. In some embodiments, the AKT inhibitor inhibits one or more activities of AKT (e.g., AKT1). In some embodiments, the anti-cancer therapy/AKT inhibitor is (a) a small molecule that inhibits one or more enzymatic activities of AKT1, (b) an antibody that inhibits one or more activities of AKT1 (e.g., by binding to and inhibiting one or more activities of AKT1, binding to and inhibiting expression of AKT1, and/or binding to and inhibiting one or more activities of a cell expressing AKT1, such as by inducing antibody-dependent cellular cytotoxicity, ADCC, or phagocytosis, ADCP), or (c) a nucleic acid that inhibits expression of AKT1 (e.g., an antisense oligonucleotide, miRNA, siRNA, morpholino, CRISPR-based therapeutic, and the like). In some embodiments, the AKT1 inhibitor is a small molecule inhibitor of AKT1 (e.g., a competitive or non-competitive inhibitor). Non-limiting examples of AKT1 inhibitors include GSK690693, GSK2141795 (uprosertib), GSK2110183 (afuresertib), AZD5363, GDC-0068 (ipatasertib), AT7867, CCT128930, MK-2206, BAY 1125976, AKT1 and AKT2-IN-1, perifosine, and VIII, as well as pharmaceutically acceptable salts thereof. In some embodiments, the AKT1 inhibitor is a pan-Akt inhibitor.

In some embodiments, the anti-cancer therapy is a hedgehog (Hh) inhibitor. In some embodiments, the anti-cancer therapy/Hh inhibitor is (a) a small molecule that inhibits one or more enzymatic activities of Hh, (b) an antibody that inhibits one or more activities of Hh (e.g., by binding to and inhibiting one or more activities of Hh, binding to and inhibiting expression of Hh, and/or binding to and inhibiting one or more activities of a cell expressing Hh, such as by inducing antibody-dependent cellular cytotoxicity, ADCC, or phagocytosis, ADCP), or (c) a nucleic acid that inhibits expression of Hh (e.g., an antisense oligonucleotide, miRNA, siRNA, morpholino, CRISPR-based therapeutic, and the like). In some embodiments, the Hh inhibitor is a small molecule inhibitor of Hh (e.g., a competitive or non-competitive inhibitor). Non-limiting examples of Hh inhibitors include sonidegib, vismodegib, erismodegib, saridegib, BMS833923, PF-04449913, and LY2940680, as well as pharmaceutically acceptable salts thereof.

In some embodiments, the anti-cancer therapy comprises a heat shock protein (HSP) inhibitor, a MYC inhibitor, an HDAC inhibitor, an immunotherapy, a neoantigen, a vaccine, or a cellular therapy.

In some embodiments, the anti-cancer therapy comprises one or more of an immune checkpoint inhibitor, a chemotherapy, a VEGF inhibitor, an Integrin β3 inhibitor, a statin, an EGFR inhibitor, an mTOR inhibitor, a PI3K inhibitor, a MAPK inhibitor, or a CDK4/6 inhibitor.

In some embodiments, the anti-cancer therapy comprises a kinase inhibitor. In some embodiments, the methods provided herein comprise administering to the individual a kinase inhibitor, e.g., in combination with another anti-cancer therapy. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). In some embodiments, the kinase inhibitor is an ALK kinase inhibitor, e.g., as described in examples 3-39 of WO2005016894, which is incorporated herein by reference.

In some embodiments, the anti-cancer therapy comprises a heat shock protein (HSP) inhibitor. In some embodiments, the methods provided herein comprise administering to the individual an HSP inhibitor, e.g., in combination with another anti-cancer therapy. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor. In some embodiments, the methods provided herein comprise administering to the individual a MYC inhibitor, e.g., in combination with another anti-cancer therapy. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor. In some embodiments, the methods provided herein comprise administering to the individual an HDAC inhibitor, e.g., in combination with another anti-cancer therapy. In some embodiments, the HDAC inhibitor is belinostat (PXD101, Beleodaq®), SAHA (vorinostat, suberoylanilide hydroxamine, Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, CIA, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor. In some embodiments, the methods provided herein comprise administering to the individual a VEGF inhibitor, e.g., in combination with another anti-cancer therapy. In some embodiments, the VEGF inhibitor is Bevacizumab (Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin β3 inhibitor. In some embodiments, the methods provided herein comprise administering to the individual an integrin β3 inhibitor, e.g., in combination with another anti-cancer therapy. In some embodiments, the integrin β3 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor. In some embodiments, the methods provided herein comprise administering to the individual an αIIbβ3 integrin inhibitor, e.g., in combination with another anti-cancer therapy. In some embodiments, the αIIbβ3 integrin inhibitor is abciximab, eptifibatide (Integrilin®), or tirofiban (Aggrastat®).

In some embodiments, the anti-cancer therapy comprises a statin or a statin-based agent. In some embodiments, the methods provided herein comprise administering to the individual a statin or a statin-based agent, e.g., in combination with another anti-cancer therapy. In some embodiments, the statin or statin-based agent is simvastatin, atorvastatin, fluvastatin, pitavastatin, pravastatin, rosuvastatin, or cerivastatin.

In some embodiments, the anti-cancer therapy comprises a MAPK inhibitor. In some embodiments, the methods provided herein comprise administering to the individual a MAPK inhibitor, e.g., in combination with another anti-cancer therapy. In some embodiments, the MAPK inhibitor is SB203580, SKF-86002, BIRB-796, SC-409, RJW-67657, BIRB-796, VX-745, R03201195, SB-242235, or MW181.

In some embodiments, the anti-cancer therapy comprises an EGFR inhibitor. In some embodiments, the methods provided herein comprise administering to the individual an EGFR inhibitor, e.g., in combination with another anti-cancer therapy. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, or erlotinib. In some embodiments, the EGFR inhibitor is gefitinib or cetuximab.

In some embodiments, the anti-cancer therapy comprises a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy. In some embodiments, the methods provided herein comprise administering to the individual a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy, e.g., in combination with another anti-cancer therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen expressed by a cancer of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et al, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104(1): 14-21, 2013). In some embodiments, such cancer vaccines augment the anti-cancer response.

In some embodiments, the cancer vaccine comprises a polynucleotide that encodes a neoantigen, e.g., a neoantigen expressed by a cancer of the disclosure. In some embodiments, the cancer vaccine comprises DNA that encodes a neoantigen, e.g., a neoantigen expressed by a cancer of the disclosure. In some embodiments, the cancer vaccine comprises RNA that encodes a neoantigen, e.g., a neoantigen expressed by a cancer of the disclosure. In some embodiments, the cancer vaccine comprises a polynucleotide that encodes a neoantigen, e.g., a neoantigen expressed by a cancer of the disclosure. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543). prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TGO2 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +−strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and birnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B 18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises an immune checkpoint inhibitor. In some embodiments, the methods provided herein comprise administering to the individual an immune checkpoint inhibitor, e.g., in combination with another anti-cancer therapy. In some embodiments, the methods provided herein comprise administering to an individual an effective amount of an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GALS, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardo11, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is one or more of MDX-1 106 (nivolumab), MK-3475 (pembrolizumab, Keytruda®), MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (1E1308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZMO09, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the anti-cancer therapy comprises an immunoregulatory molecule or a cytokine. In some embodiments, the methods provided herein comprise administering to the individual an immunoregulatory molecule or a cytokine, e.g., in combination with another anti-cancer therapy. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, 1309, IL-8, GCP-2 Groa, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is monovalent and/or monospecific. In some embodiments, the immune checkpoint inhibitor is multivalent and/or multispecific.

In some embodiments, the anti-cancer therapy comprises a nucleic acid molecule, such as a dsRNA, an siRNA, or an shRNA. In some embodiments, the methods provided herein comprise administering to the individual a nucleic acid molecule, such as a dsRNA, an siRNA, or an shRNA, e.g., in combination with another anti-cancer therapy. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. Methods for designing, optimizing, producing, and using dsRNAs, siRNAs, or shRNAs, are known in the art.

In some embodiments, the anti-cancer therapy comprises a chemotherapy. In some embodiments, the methods provided herein comprise administering to the individual a chemotherapy, e.g., in combination with another anti-cancer therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor. In some embodiments, the methods provided herein comprise administering to the individual a kinase inhibitor, e.g., in combination with another anti-cancer therapy. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR3, FGFR4, CSF1R, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent. In some embodiments, the methods provided herein comprise administering to the individual an anti-angiogenic agent, e.g., in combination with another anti-cancer therapy. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy. In some embodiments, the methods provided herein comprise administering to the individual an anti-DNA repair therapy, e.g., in combination with another anti-cancer therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer. In some embodiments, the methods provided herein comprise administering to the individual a radiosensitizer, e.g., in combination with another anti-cancer therapy. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent. In some embodiments, the methods provided herein comprise administering to the individual an anti-inflammatory agent, e.g., in combination with another anti-cancer therapy. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the anti-cancer therapy comprises an anti-hormonal agent. In some embodiments, the methods provided herein comprise administering to the individual an anti-hormonal agent, e.g., in combination with another anti-cancer therapy. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent. In some embodiments, the methods provided herein comprise administering to the individual an antimetabolite chemotherapeutic agent, e.g., in combination with another anti-cancer therapy. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent. In some embodiments, the methods provided herein comprise administering to the individual a platinum-based chemotherapeutic agent, e.g., in combination with another anti-cancer therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein, and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 1 19, Marcel Dekker, 2002.

Formulations to be used for in vivo administration are sterile. This is readily accomplished by filtration through sterile filtration membranes or other methods known in the art.

In some embodiments, the anti-cancer therapy is administered as a monotherapy. In some embodiments, the anti-cancer therapy is administered in combination with one or more additional anti-cancer therapies or treatments. In some embodiments, the one or more additional anti-cancer therapies or treatments include one or more anti-cancer therapies described herein. In some embodiments, the methods of the present disclosure comprise administration of any combination of any of the anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy. In some embodiments, the anti-cancer therapy for use in any of the methods described herein (e.g., as monotherapy or in combination with another therapy or treatment) is an anti-cancer therapy or treatment described by Pietrantonio et al., J Natl Cancer Inst (2017) 109(12) and/or by Wang et al., Cancers (2020) 12(2):426, which are hereby incorporated by reference.

Kits

Also provided herein are kits for improving sequencing analysis and/or extracting nucleic acids according to any one of the methods described herein.

In some embodiments, the kit includes reagents and instructions for performing the methods of the present disclosure. In some embodiments, the kit comprises reagents for extracting a sample from the tissue, e.g., a needle as described herein. In some embodiments, the kit further comprises reagents for extracting one or more nucleic acids from the sample.

Also provided herein are kits for detecting a biomarker (e.g., LOH, HLA LOH, PTEN LOF, TMB, and/or homozygous single exon loss) nucleic acid molecule of the disclosure, e.g., in the nucleic acids extracted from a sample extracted from a tissue, as described herein. In some embodiments, a kit provided herein comprises a reagent (e.g., one or more oligonucleotides, primers, probes or baits of the present disclosure) for detecting a biomarker (e.g., LOH, HLA LOH, PTEN LOF, TMB, and/or homozygous single exon loss) nucleic acid molecule provided herein. In some embodiments, the kit comprises a reagent (e.g., one or more oligonucleotides, primers, probes or baits of the present disclosure) for detecting a wild-type counterpart of a biomarker (e.g., LOH, HLA LOH, PTEN LOF, TMB, and/or homozygous single exon loss) nucleic acid molecule provided herein. In some embodiments, the reagent comprises one or more oligonucleotides, primers, probes or baits of the present disclosure capable of hybridizing to a biomarker (e.g., LOH, HLA LOH, PTEN LOF, TMB, and/or homozygous single exon loss) nucleic acid molecule provided herein, or to a wild-type counterpart of a biomarker (e.g., LOH, HLA LOH, PTEN LOF, TMB, and/or homozygous single exon loss) nucleic acid molecule provided herein. In some embodiments, the reagent comprises one or more oligonucleotides, primers, probes or baits of the present disclosure capable of distinguishing a biomarker (e.g., LOH, HLA LOH, PTEN LOF, TMB, and/or homozygous single exon loss) nucleic acid molecule provided herein from a wild-type counterpart of the biomarker (e.g., LOH, HLA LOH, PTEN LOF, TMB, and/or homozygous single exon loss) nucleic acid molecule provided herein. In some embodiments, the kit is for use according to any method of detecting biomarker (e.g., LOH, HLA LOH, PTEN LOF, TMB, and/or homozygous single exon loss) nucleic acid molecules known in the art or described herein, such as sequencing, PCR, in situ hybridization methods, a nucleic acid hybridization assay, an amplification-based assay, a PCR-RFLP assay, real-time PCR, sequencing, next-generation sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting a biomarker (e.g., LOH, HLA LOH, PTEN LOF, TMB, and/or homozygous single exon loss) nucleic acid molecule of the disclosure, e.g., using one or more oligonucleotides, primers, probes or baits of the present disclosure.

### EXEMPLARY EMBODIMENTS

The following exemplary embodiments are representative of some aspects of the invention:

Exemplary Embodiment 1: A method of improving sequencing analysis, wherein the method comprises: a) identifying a target region comprising tumor cells of interest in a tissue; b) extracting a sample from the tissue; c) identifying the location of the sample in the tissue; and d) if the location of the sample overlaps with the target region comprising tumor cells of interest, extracting one or more nucleic acids from the sample.

Exemplary Embodiment 2: A method of extracting nucleic acids, wherein the method comprises: a) identifying a target region comprising tumor cells of interest in a tissue; b) extracting a sample from the tissue; c) identifying the location of the sample in the tissue; and d) if the location of the sample overlaps with the target region comprising tumor cells of interest, extracting one or more nucleic acids from the sample.

Exemplary Embodiment 3: The method of any one of the preceding embodiments, wherein if the location of the sample does not overlap with the target region comprising tumor cells of interest, steps b) and c) are repeated.

Exemplary Embodiment 4: The method of any one of the preceding embodiments, wherein step b) comprises extracting the sample using a needle.

Exemplary Embodiment 5: The method of embodiment 4, wherein the needle is punched through the tissue, thereby extracting the sample.

Exemplary Embodiment 6: The method of embodiment 4 or embodiment 5, wherein the needle is a disposable needle.

Exemplary Embodiment 7: The method of any one of embodiments 4-6, wherein the needle is a 13 gauge needle, 14 gauge needle, a 15 gauge needle, a 16 gauge needle, a 17 gauge needle, a 18 gauge needle, a 19 gauge needle, a 20 gauge needle, or a 21 gauge needle.

Exemplary Embodiment 8: The method of any one of embodiments 4-7, wherein the sample extracted from the tissue is about 0.8, 0.9, 1.0, 1.2, 1.4, 1.6, 1.8, 2.0, or 2.3 mm in diameter.

Exemplary Embodiment 9: The method of any one embodiments 1-3, wherein step b) comprises extracting the sample using laser microdissection (LMD) or a razor blade.

Exemplary Embodiment 10: The method of any one of the preceding embodiments, wherein step c) comprises preparing a slide of a section of the tissue.

Exemplary Embodiment 11: The method of embodiment 10, wherein the section of the tissue is stained.

Exemplary Embodiment 12: The method of embodiment 10 or embodiment 11, wherein the section of the tissue is Haematoxylin and Eosin (H&E) stained.

Exemplary Embodiment 13: The method of any one of the preceding embodiments, wherein step c) is performed by visual inspection.

Exemplary Embodiment 14: The method of embodiments 1-12, wherein step c) is performed by a computer system.

Exemplary Embodiment 15: The method of embodiments 1-12, wherein step c) is performed using an image analysis system.

Exemplary Embodiment 16: A method of improving sequencing analysis, wherein the method comprises: a) providing a tissue comprising tumor cells of interest; b) extracting a sample from the tissue; c) assessing the level of enrichment of the tumor cells of interest in the sample and in the remaining tissue; and d) if the level of enrichment of tumor cells of interest in the sample exceeds the level of tumor cells of interest in the remaining tissue, extracting one or more nucleic acids from the sample.

Exemplary Embodiment 17: A method of extracting nucleic acids, wherein the method comprises: a) providing a tissue comprising tumor cells of interest; b) extracting a sample from the tissue; c) assessing the level of enrichment of the tumor cells of interest in the sample and in the remaining tissue; and d) if the level of enrichment of tumor cells of interest in the sample exceeds the level of tumor cells of interest in the remaining tissue, extracting one or more nucleic acids from the sample.

Exemplary Embodiment 18: The method of embodiment 16 or embodiment 17, wherein if the level of enrichment of tumor cells of interest in the sample does not exceed the level of tumor cells of interest in the remaining tissue, steps b) and c) are repeated.

Exemplary Embodiment 19: The method of any one of the preceding embodiments, wherein the level of enrichment of tumor cells of interest in the sample is at least 1-fold, 1.1-fold, 1.2-fold, 1.3-fold, 1.4-fold, 1.5-fold, 1.6-fold, 1.7-fold, 1.8-fold, 1.9-fold, or 2-fold higher than the level of tumor cells of interest in the remaining tissue.

Exemplary Embodiment 20: The method of any one of the preceding embodiments, wherein the tissue is from an individual known to have cancer or suspected of having cancer.

Exemplary Embodiment 21: The method of any one of the preceding embodiments, wherein the tissue is from a biopsy, optionally a tumor biopsy.

Exemplary Embodiment 22: The method of any one of the preceding embodiments, wherein the tissue is a fixed tissue.

Exemplary Embodiment 23: The method of embodiment 22, wherein the fixed tissue is selected from the group consisting of a formalin-fixed tissue, an ethanol-fixed tissue, and a methanol-fixed tissue.

Exemplary Embodiment 24: The method of any one of the preceding embodiments, wherein the tissue is embedded in an embedding agent.

Exemplary Embodiment 25: The method of embodiment 24, wherein the embedding agent is resin or paraffin.

Exemplary Embodiment 26: The method of any one of the preceding embodiments, wherein the tissue is a formalin-fixed paraffin-embedded (FFPE) tissue.

Exemplary Embodiment 27: The method of any one of embodiments 1-21, wherein the tissue is a cryopreserved tissue.

Exemplary Embodiment 28: The method of any one of embodiments 1-21, wherein the tissue is a fresh-frozen tissue.

Exemplary Embodiment 29: The method of embodiment 28, wherein the fresh-frozen tissue is frozen in an optimal cutting temperature (OCT) compound.

Exemplary Embodiment 30: The method of any one of the preceding embodiments, wherein step b) further comprises inspecting the sample, and optionally removing any excess tissue from the sample.

Exemplary Embodiment 31: The method of any one of embodiments 24-26, wherein step b) further comprises inspecting the sample, and optionally removing any excess embedding agent from the sample.

Exemplary Embodiment 32: The method of any one of the preceding embodiments, wherein the method further comprises subjecting the one or more nucleic acids extracted from the sample to further processing, optionally wherein the further processing comprises digestion, DnaX treatment, gel electrophoresis, and/or quantification.

Exemplary Embodiment 33: The method of any one of the preceding embodiments, wherein the one or more nucleic acids extracted from the sample comprise RNA and/or DNA.

Exemplary Embodiment 34: The method of any one of the preceding embodiments, wherein the one or more nucleic acids extracted from the sample are analyzed by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping.

Exemplary Embodiment 35: The method of any one of the preceding embodiments, wherein the one or more nucleic acids extracted from the sample are analyzed by next-generation sequencing.

Exemplary Embodiment 36: The method of any one of the preceding embodiments, wherein the method further comprises: e) optionally, ligating one or more adaptors onto one or more nucleic acids extracted from the sample; d) optionally, amplifying nucleic acids from the ligated nucleic acids; f) optionally, capturing a plurality of nucleic acids corresponding to one or more genes of interest; g) sequencing, by a sequencer, the plurality of nucleic acids to obtain a plurality of sequence reads corresponding to the one or more genes of interest; h) analyzing the plurality of sequence reads; and i) based on the analysis, detecting one or more mutations in a gene of interest.

Exemplary Embodiment 37: The method of embodiment 36, wherein the plurality of nucleic acids corresponding to the one or more genes of interest is captured from the amplified nucleic acids by hybridization with a bait molecule.

Exemplary Embodiment 38: The method of embodiment 36, wherein prior to step e) the one or more nucleic acids extracted from the sample are fragmented, optionally wherein the one or more nucleic acids extracted from the sample are fragmented by sonication.

Exemplary Embodiment 39: he method of embodiment 38, wherein the fragmented one or more nucleic acids extracted from the sample are end-repaired.

Exemplary Embodiment 40: The method of embodiment 39, wherein the end-repaired, fragmented one or more nucleic acids extracted from the sample are dA-tailed or dT-tailed.

Exemplary Embodiment 41: The method of any one of the preceding embodiments, wherein the method further comprises detecting loss-of-heterozygosity (LOH) of one or more genes of interest in the sample.

Exemplary Embodiment 42: The method of embodiment 41, wherein the method further comprises detecting LOH of ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEGS, NNAT, GNAS, and/or GATA5 in the sample.

Exemplary Embodiment 43: The method of embodiment 41, wherein the method further comprises detecting LOH of a human leukocyte antigen (HLA) gene in the sample.

Exemplary Embodiment 44: The method of embodiment 43, wherein the method further comprises: ligating one or more adaptors onto one or more of the one or more nucleic acids extracted from the sample; amplifying the ligated nucleic acids; capturing a plurality of nucleic acids corresponding to the HLA gene from the amplified nucleic acids using a bait molecule; sequencing the captured nucleic acids to obtain a plurality of sequence reads corresponding to the HLA gene; fitting, by one or more processors, one or more values associated with one or more of the plurality of sequence reads to a model; and based on the model, detecting LOH of the HLA gene and a relative binding propensity for an HLA allele of the HLA gene.

Exemplary Embodiment 45: The method of embodiment 44, wherein LOH of the HLA gene and relative binding propensity for an HLA allele of the HLA gene are detected by: a) obtaining an observed allele frequency for an HLA allele, wherein observed allele frequency corresponds to frequency of nucleic acid(s) encoding at least a portion of the HLA allele as detected among the plurality of sequence reads corresponding to the HLA gene; b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) applying an objective function to measure a difference between the relative binding propensity and the observed allele frequency of the HLA allele; d) applying an optimization model to minimize the objective function; e) determining an adjusted allele frequency of the HLA allele based on the optimization model and the observed allele frequency; and f) determining that LOH has occurred when the adjusted allele frequency of the HLA allele is less than a predetermined threshold.

Exemplary Embodiment 46: The method of embodiment 44 or embodiment 45, further comprising, based at least in part on detection of LOH of the HLA gene, administering an effective amount of a treatment other than an immune checkpoint inhibitor (ICI) to the individual.

Exemplary Embodiment 47: The method of embodiment 44 or embodiment 45, further comprising, based at least in part on detection of LOH of the HLA gene, recommending a treatment other than an immune checkpoint inhibitor (ICI).

Exemplary Embodiment 48: The method of embodiment 44 or embodiment 45, further comprising: detecting, or acquiring knowledge of, a high tumor mutational burden (TMB) in the sample.

Exemplary Embodiment 49: The method of embodiment 48, further comprising, based at least in part on detection of LOH of the HLA gene and high TMB, administering an effective amount of an immune checkpoint inhibitor (ICI) to the individual.

Exemplary Embodiment 50: The method of embodiment 48, further comprising, based at least in part on detection of LOH of the HLA gene and high TMB, recommending a treatment comprising an immune checkpoint inhibitor (ICI) to the individual.

Exemplary Embodiment 51: The method of any one of embodiments 43-50, wherein the HLA gene is a human HLA-A, HLA-B, or HLA-C gene.

Exemplary Embodiment 52: The method of any one of embodiments 48-51, wherein the TMB is determined based on a number of non-driver somatic coding mutations per megabase of genome sequenced.

Exemplary Embodiment 53: The method of any one of embodiments 1-40, wherein the method further comprises detecting a loss-of-function mutation in a phosphatase and tensin homolog (PTEN) gene in the sample.

Exemplary Embodiment 54: The method of embodiment 53, wherein the loss-of-function mutation in a PTEN gene comprises one or more of an insertion, deletion or substitution of one or more nucleotides, a genomic rearrangement, an alteration in a promoter, a gene fusion, or a copy number alteration.

Exemplary Embodiment 55: The method of embodiment 53 or embodiment 54, wherein the loss-of-function mutation in a PTEN gene is detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 56: The method of any one of embodiments 1-40, wherein the method further comprises measuring the level of tumor mutational burden (TMB) in the sample.

Exemplary Embodiment 57: The method of embodiment 56, wherein a TMB of at least about 10 mutations/megabase (Mb) or at least about 20 mut/Mb is detected.

Exemplary Embodiment 58: The method of embodiment 56 or embodiment 57, wherein TMB is measured in the one or more nucleic acids from the sample by whole exome sequencing, whole genome sequencing, or gene-targeted sequencing.

Exemplary Embodiment 59: The method of embodiment 58, wherein TMB is measured on about 0.80 Mb of sequenced DNA.

Exemplary Embodiment 60: The method of embodiment 58, wherein TMB is measured on between about 0.83 Mb and about 1.14 Mb of sequenced DNA.

Exemplary Embodiment 61: The method of embodiment 58, wherein TMB is measured on about 1.1 Mb of sequenced DNA.

Exemplary Embodiment 62: The method of embodiment 58, wherein TMB is measured on up to about 1.1 Mb of sequenced DNA.

Exemplary Embodiment 63: The method of any one of embodiments 1-40, wherein the method further comprises detecting homozygous single exon loss in the sample.

Exemplary Embodiment 64: The method of embodiment 63, wherein the homozygous single exon loss is detected in the one or more nucleic acids from the sample by whole exome sequencing, whole genome sequencing, or gene-targeted sequencing.

Exemplary Embodiment 65: A system comprising: a memory configured to store one or more program instructions; and one or more processors configured to execute the one or more program instructions, wherein the one or more program instructions when executed by the one or more processors are configured to: (a) obtain a plurality of sequence reads of one or more nucleic acids, wherein the one or more nucleic acids are derived from a sample extracted from a tissue according to any one of the methods of the preceding embodiments; (b) analyze the plurality of sequence reads for the presence of LOH of one or more genes of interest, LOH of a HLA gene, a loss-of-function mutation in a PTEN gene, TMB of at least about 10 mut/Mb or at least about 20 mut/Mb, and/or homozygous single exon loss; and (c) detect, based on the analyzing, LOH of one or more genes of interest, LOH of a HLA gene, a loss-of-function mutation in a PTEN gene, TMB of at least about 10 mut/Mb or at least about 20 mut/Mb, and/or homozygous single exon loss in the sample.

Exemplary Embodiment 66: A method of identifying an individual having cancer who may benefit from a treatment comprising an anti-cancer therapy, the method comprising detecting LOH of one or more genes of interest in a sample extracted from a tissue from the individual according to the method of embodiment 41 or embodiment 42, wherein the presence of LOH of one or more genes of interest in a sample extracted from a tissue identifies the individual as one who may or may not benefit from the anti-cancer therapy.

Exemplary Embodiment 67: A method of detecting the presence or absence of a cancer in an individual, the method comprising detecting the presence or absence of LOH of one or more genes of interest in a sample extracted from a tissue from the individual according to the method of embodiment 41 or embodiment 42.

Exemplary Embodiment 68: A method of selecting a therapy for an individual having cancer, the method comprising detecting LOH of one or more genes of interest in a sample extracted from a tissue from the individual according to the method of embodiment 41 or embodiment 42, wherein the presence of LOH of one or more genes of interest in a sample extracted from a tissue from the individual identifies the individual as one who may benefit from a treatment comprising an anti-cancer therapy.

Exemplary Embodiment 69: A method of identifying one or more treatment options for an individual having cancer, the method comprising: (a) detecting LOH of one or more genes of interest in a sample extracted from a tissue from the individual according to the method of embodiment 41 or embodiment 42; and (b) generating a report comprising one or more treatment options identified for the individual based at least in part on the presence of the LOH of one or more genes of interest in the sample extracted from a tissue from the individual, wherein the one or more treatment options comprise an anti-cancer therapy.

Exemplary Embodiment 70: A method of identifying one or more treatment options for an individual having cancer, the method comprising: (a) acquiring knowledge of LOH of one or more genes of interest in a sample extracted from a tissue from the individual according to the method of embodiment 41 or embodiment 42; and (b) generating a report comprising one or more treatment options identified for the individual based at least in part on said knowledge, wherein the one or more treatment options comprise an anti-cancer therapy.

Exemplary Embodiment 71: A method of selecting or not selecting a treatment for an individual having cancer, comprising acquiring knowledge of LOH of one or more genes of interest in a sample extracted from a tissue from the individual having cancer according to the method of embodiment 41 or embodiment 42, wherein responsive to the acquisition of said knowledge: (i) the individual is classified as a candidate to receive treatment with an anti-cancer therapy, or the individual is not classified as a candidate to receive treatment with an anti-cancer therapy; and/or (ii) the individual is identified as likely to respond to a treatment that comprises an anti-cancer therapy, or the individual is identified as unlikely to respond to a treatment that comprises an anti-cancer therapy.

Exemplary Embodiment 72: A method of treating or delaying progression of cancer, comprising: (a) acquiring knowledge of LOH of one or more genes of interest in a sample extracted from a tissue from an individual according to the method of embodiment 41 or embodiment 42; and (b) responsive to said knowledge, administering to the individual an effective amount of a treatment that comprises an anti-cancer therapy.

Exemplary Embodiment 73: A method of treating or delaying progression of cancer, comprising, responsive to acquiring knowledge of LOH of one or more genes of interest in a sample extracted from a tissue from an individual according to the method of embodiment 41 or embodiment 42, administering to the individual an effective amount of a treatment that comprises an anti-cancer therapy.

Exemplary Embodiment 74: A method of treating or delaying progression of cancer, comprising: (a) detecting LOH of one or more genes of interest in a sample extracted from a tissue from an individual according to the method of embodiment 41 or embodiment 42; and (b) administering to the individual an effective amount of a treatment that comprises an anti-cancer therapy.

Exemplary Embodiment 75: A method of diagnosing/assessing LOH of one or more genes of interest in a cancer in an individual, the method comprising: (a) detecting LOH of one or more genes of interest in a sample extracted from a tissue from the individual according to the method of embodiment 41 or embodiment 42; and (b) providing an assessment of the LOH of one or more genes of interest.

Exemplary Embodiment 76: A method of detecting LOH of one or more genes of interest, the method comprising detecting LOH of one or more genes of interest in a sample extracted from a tissue from an individual according to the method of embodiment 41 or embodiment 42.

Exemplary Embodiment 77: A method of identifying an individual having cancer who may benefit from a treatment comprising an anti-cancer therapy, the method comprising detecting LOH of an HLA gene in a sample extracted from a tissue in an individual according to the method of any one of embodiments 41 or 43-52, wherein the presence of LOH of an HLA gene in a sample extracted from the tissue identifies the individual as one who may or may not benefit from the anti-cancer therapy.

Exemplary Embodiment 78: A method of detecting the presence or absence of a cancer in an individual, the method comprising detecting the presence or absence of LOH of an HLA gene in a sample extracted from a tissue from the individual according to the method of any one of embodiments 41 or 43-52.

Exemplary Embodiment 79: A method of selecting a therapy for an individual having cancer, the method comprising detecting LOH of an HLA gene in a sample extracted from a tissue from the individual according to the method of any one of embodiments 41 or 43-52, wherein the presence of LOH of an HLA gene in a sample extracted from a tissue from the individual identifies the individual as one who may benefit from a treatment comprising an anti-cancer therapy.

Exemplary Embodiment 80: A method of identifying one or more treatment options for an individual having cancer, the method comprising: (a) detecting LOH of an HLA gene in a sample extracted from a tissue from the individual according to the method of any one of embodiments 41 or 43-52; and (b) generating a report comprising one or more treatment options identified for the individual based at least in part on the presence of the one or more mutations in the LOH of an HLA gene in the sample, wherein the one or more treatment options comprise an anti-cancer therapy.

Exemplary Embodiment 81: A method of identifying one or more treatment options for an individual having cancer, the method comprising: (a) acquiring knowledge of LOH of an HLA gene in a sample extracted from a tissue from the individual according to the method of any one of embodiments 41 or 43-52; and (b) generating a report comprising one or more treatment options identified for the individual based at least in part on said knowledge, wherein the one or more treatment options comprise an anti-cancer therapy.

Exemplary Embodiment 82: A method of selecting or not selecting a treatment for an individual having cancer, comprising acquiring knowledge of LOH of an HLA gene in a sample extracted from a tissue from the individual having cancer according to the method of any one of embodiments 41 or 43-52, wherein responsive to the acquisition of said knowledge: (i) the individual is classified as a candidate to receive treatment with an anti-cancer therapy, or the individual is not classified as a candidate to receive treatment with an anti-cancer therapy; and/or (ii) the individual is identified as likely to respond to a treatment that comprises an anti-cancer therapy, or the individual is identified as unlikely to respond to a treatment that comprises an anti-cancer therapy.

Exemplary Embodiment 83: A method of treating or delaying progression of cancer, comprising: (a) acquiring knowledge of LOH of an HLA gene in a sample extracted from a tissue from an individual according to the method of any one of embodiments 41 or 43-52; and (b) responsive to said knowledge, administering to the individual an effective amount of a treatment that comprises an anti-cancer therapy.

Exemplary Embodiment 84: A method of treating or delaying progression of cancer, comprising, responsive to acquiring knowledge of LOH of an HLA gene in a sample extracted from a tissue from an individual according to the method of any one of embodiments 41 or 43-52, administering to the individual an effective amount of a treatment that comprises an anti-cancer therapy.

Exemplary Embodiment 85: A method of treating or delaying progression of cancer, comprising: (a) detecting LOH of an HLA gene in a sample extracted from a tissue from an individual according to the method of any one of cl embodiments aims 41 or 43-52; and (b) administering to the individual an effective amount of a treatment that comprises an anti-cancer therapy.

Exemplary Embodiment 86: A method of diagnosing/assessing LOH of one or more genes of interest in a cancer in an individual, the method comprising: (a) detecting LOH of an HLA gene in a sample extracted from a tissue from an individual according to the method of any one of embodiments 41 or 43-52; and (b) providing an assessment of the LOH of an HLA gene.

Exemplary Embodiment 87: A method of detecting LOH of an HLA gene, the method comprising detecting LOH of an HLA gene in a sample extracted from a tissue from an individual according to the method of any one of embodiments 41 or 43-52.

Exemplary Embodiment 88: A method of identifying an individual having cancer who may benefit from a treatment comprising an anti-cancer therapy, the method comprising detecting a loss-of-function mutation in a PTEN gene in a sample extracted from a tissue from an individual according to the method of any one of embodiments 53-55, wherein the presence of a loss-of-function mutation in a PTEN gene in a sample extracted from a tissue identifies the individual as one who may or may not benefit from the anti-cancer therapy.

Exemplary Embodiment 89: A method of detecting the presence or absence of a cancer in an individual, the method comprising detecting the presence or absence of a loss-of-function mutation in a PTEN gene in a sample extracted from a tissue from the individual according to the method of any one of embodiments 53-55.

Exemplary Embodiment 90: A method of selecting a therapy for an individual having cancer, the method comprising detecting a loss-of-function mutation in a PTEN gene in a sample extracted from a tissue from an individual according to the method of any one of embodiments 53-55, wherein the presence of a loss-of-function mutation in a PTEN gene in a sample extracted from a tissue from the individual identifies the individual as one who may benefit from a treatment comprising an anti-cancer therapy.

Exemplary Embodiment 91: A method of identifying one or more treatment options for an individual having cancer, the method comprising: (a) detecting a loss-of-function mutation in a PTEN gene in a sample extracted from a tissue from the individual according to the method of any one of embodiments 53-55; and (b) generating a report comprising one or more treatment options identified for the individual based at least in part on the presence of the one or more mutations in the a loss-of-function mutation in a PTEN gene in the sample, wherein the one or more treatment options comprise an anti-cancer therapy.

Exemplary Embodiment 92: A method of identifying one or more treatment options for an individual having cancer, the method comprising: (a) acquiring knowledge of a loss-of-function mutation in a PTEN gene in a sample extracted from a tissue from the individual according to the method of any one of embodiments 53-55; and (b) generating a report comprising one or more treatment options identified for the individual based at least in part on said knowledge, wherein the one or more treatment options comprise an anti-cancer therapy.

Exemplary Embodiment 93: A method of selecting or not selecting a treatment for an individual having cancer, comprising acquiring knowledge of a loss-of-function mutation in a PTEN gene in a sample extracted from a tissue from the individual having cancer according to the method of any one of embodiments 53-55, wherein responsive to the acquisition of said knowledge: (i) the individual is classified as a candidate to receive treatment with an anti-cancer therapy, or the individual is not classified as a candidate to receive treatment with an anti-cancer therapy; and/or (ii) the individual is identified as likely to respond to a treatment that comprises an anti-cancer therapy, or the individual is identified as unlikely to respond to a treatment that comprises an anti-cancer therapy.

Exemplary Embodiment 94: A method of treating or delaying progression of cancer, comprising: (a) acquiring knowledge of a loss-of-function mutation in a PTEN gene in a sample extracted from a tissue from an individual according to the method of any one of embodiments 53-55; and (b) responsive to said knowledge, administering to the individual an effective amount of a treatment that comprises an anti-cancer therapy.

Exemplary Embodiment 95: A method of treating or delaying progression of cancer, comprising, responsive to acquiring knowledge of a loss-of-function mutation in a PTEN gene in a sample extracted from a tissue from an individual according to the method of any one of embodiments 53-55, administering to the individual an effective amount of a treatment that comprises an anti-cancer therapy.

Exemplary Embodiment 96: A method of treating or delaying progression of cancer, comprising: (a) detecting a loss-of-function mutation in a PTEN gene in a sample extracted from a tissue from an individual according to the method of any one of embodiments 53-55; and (b) administering to the individual an effective amount of a treatment that comprises an anti-cancer therapy.

Exemplary Embodiment 97: A method of diagnosing/assessing a loss-of-function mutation in a PTEN gene in a cancer in an individual, the method comprising: (a) detecting a loss-of-function mutation in a PTEN gene in a sample extracted from a tissue from the individual according to the method of any one of embodiments 53-55; and (b) providing an assessment of the loss-of-function mutation in a PTEN gene.

Exemplary Embodiment 98: A method of detecting a loss-of-function mutation in a PTEN gene, the method comprising detecting a loss-of-function mutation in a PTEN gene in a sample extracted from a tissue from an individual according to the method of any one of embodiments 53-55.

Exemplary Embodiment 99: A method of identifying an individual having cancer who may benefit from a treatment comprising an anti-cancer therapy, the method comprising detecting the level of TMB in a sample extracted from a tissue according to the method of any one of embodiments 56-62, wherein the level of TMB in a sample extracted from a tissue identifies the individual as one who may or may not benefit from the anti-cancer therapy.

Exemplary Embodiment 100: A method of detecting the presence or absence of a cancer in an individual, the method comprising detecting the level of TMB in a sample extracted from a tissue according to the method of any one of embodiments 56-62.

Exemplary Embodiment 101: A method of selecting a therapy for an individual having cancer, the method comprising detecting the level of TMB in a sample extracted from a tissue from an individual according to the method of any one of embodiments 56-62, wherein the level of TMB in a sample extracted from a tissue from the individual identifies the individual as one who may benefit from a treatment comprising an anti-cancer therapy.

Exemplary Embodiment 102: A method of identifying one or more treatment options for an individual having cancer, the method comprising: (a) detecting the level of TMB in a sample extracted from a tissue from the individual according to the method of any one of embodiments 56-62; and (b) generating a report comprising one or more treatment options identified for the individual based at least in part on the level of TMB in the sample, wherein the one or more treatment options comprise an anti-cancer therapy.

Exemplary Embodiment 103: A method of identifying one or more treatment options for an individual having cancer, the method comprising: (a) acquiring knowledge of the level of TMB in a sample extracted from a tissue from the individual according to the method of any one of embodiments 56-62; and (b) generating a report comprising one or more treatment options identified for the individual based at least in part on said knowledge, wherein the one or more treatment options comprise an anti-cancer therapy.

Exemplary Embodiment 104: A method of selecting or not selecting a treatment for an individual having cancer, comprising acquiring knowledge of the level of TMB in a sample extracted from a tissue from the individual having cancer according to the method of any one of embodiments 56-62, wherein responsive to the acquisition of said knowledge: (i) the individual is classified as a candidate to receive treatment with an anti-cancer therapy, or the individual is not classified as a candidate to receive treatment with an anti-cancer therapy; and/or (ii) the individual is identified as likely to respond to a treatment that comprises an anti-cancer therapy, or the individual is identified as unlikely to respond to a treatment that comprises an anti-cancer therapy.

Exemplary Embodiment 105: A method of treating or delaying progression of cancer, comprising: (a) acquiring knowledge the level of TMB in a sample extracted from a tissue from an individual according to the method of any one of embodiments 56-62; and (b) responsive to said knowledge, administering to the individual an effective amount of a treatment that comprises an anti-cancer therapy.

Exemplary Embodiment 106: A method of treating or delaying progression of cancer, comprising, responsive to acquiring knowledge of the level of TMB in a sample extracted from a tissue from an individual according to the method of any one of embodiments 56-62, administering to the individual an effective amount of a treatment that comprises an anti-cancer therapy.

Exemplary Embodiment 107: A method of treating or delaying progression of cancer, comprising: (a) detecting the level of TMB in a sample extracted from a tissue from an individual according to the method of any one of embodiments 56-62; and (b) administering to the individual an effective amount of a treatment that comprises an anti-cancer therapy.

Exemplary Embodiment 108: A method of diagnosing/assessing the level of TMB in a cancer in an individual, the method comprising: (a) detecting the level of TMB in a sample extracted from a tissue from the individual according to the method of any one of embodiments 56-62; and (b) providing an assessment of the level of TMB.

Exemplary Embodiment 109: A method of detecting the level of TMB, the method comprising detecting the level of TMB in a sample extracted from a tissue from an individual according to the method of any one of embodiments 56-62.

Exemplary Embodiment 110: A method of identifying an individual having cancer who may benefit from a treatment comprising an anti-cancer therapy, the method comprising detecting homozygous single exon loss in a sample extracted from a tissue according to the method of any one of embodiments 63-64, wherein the presence of homozygous single exon loss in a sample extracted from a tissue identifies the individual as one who may or may not benefit from the anti-cancer therapy.

Exemplary Embodiment 111: A method of detecting the presence or absence of a cancer in an individual, the method comprising detecting the presence or absence of homozygous single exon loss in a sample extracted from a tissue according to the method of any one of embodiments 63-64.

Exemplary Embodiment 112: A method of selecting a therapy for an individual having cancer, the method comprising detecting homozygous single exon loss in a sample extracted from a tissue from the individual according to the method of any one of embodiments 63-64, wherein the presence of homozygous single exon loss in a sample extracted from a tissue from the individual identifies the individual as one who may benefit from a treatment comprising an anti-cancer therapy.

Exemplary Embodiment 113: A method of identifying one or more treatment options for an individual having cancer, the method comprising: (a) detecting homozygous single exon loss extracted from a tissue from the individual according to the method of any one of embodiments 63-64; and (b) generating a report comprising one or more treatment options identified for the individual based at least in part on the presence of homozygous single exon loss in the sample, wherein the one or more treatment options comprise an anti-cancer therapy.

Exemplary Embodiment 114: A method of identifying one or more treatment options for an individual having cancer, the method comprising: (a) acquiring knowledge of homozygous single exon loss in a sample extracted from a tissue from the individual according to the method of any one of embodiments 63-64; and (b) generating a report comprising one or more treatment options identified for the individual based at least in part on said knowledge, wherein the one or more treatment options comprise an anti-cancer therapy.

Exemplary Embodiment 115: A method of selecting or not selecting a treatment for an individual having cancer, comprising acquiring knowledge of homozygous single exon loss in a sample extracted from a tissue from the individual having cancer according to the method of any one of embodiments 63-64, wherein responsive to the acquisition of said knowledge: (i) the individual is classified as a candidate to receive treatment with an anti-cancer therapy, or the individual is not classified as a candidate to receive treatment with an anti-cancer therapy; and/or (ii) the individual is identified as likely to respond to a treatment that comprises an anti-cancer therapy, or the individual is identified as unlikely to respond to a treatment that comprises an anti-cancer therapy.

Exemplary Embodiment 116: A method of treating or delaying progression of cancer, comprising: (a) acquiring knowledge of homozygous single exon loss in a sample extracted from a tissue from an individual according to the method of any one of embodiments 63-64; and (b) responsive to said knowledge, administering to the individual an effective amount of a treatment that comprises an anti-cancer therapy.

Exemplary Embodiment 117: A method of treating or delaying progression of cancer, comprising, responsive to acquiring knowledge of homozygous single exon loss in a sample extracted from a tissue from an individual according to the method of any one of embodiments 63-64, administering to the individual an effective amount of a treatment that comprises an anti-cancer therapy.

Exemplary Embodiment 118: A method of treating or delaying progression of cancer, comprising: (a) detecting homozygous single exon loss in a sample extracted from a tissue from an individual according to the method of any one of embodiments 63-64; and (b) administering to the individual an effective amount of a treatment that comprises an anti-cancer therapy.

Exemplary Embodiment 119: A method of diagnosing/assessing homozygous single exon loss in a cancer in an individual, the method comprising: (a) detecting homozygous single exon loss in a sample extracted from a tissue from the individual according to the method of any one of embodiments 63-64; and (b) providing an assessment of the homozygous single exon loss.

Exemplary Embodiment 120: A method of detecting homozygous single exon loss, the method comprising detecting homozygous single exon loss in a sample extracted from a tissue from an individual according to the method of any one of embodiments 63-64.

Exemplary Embodiment 121: The method of any one of embodiments 1-120, wherein the method reduces the incidence of tissue insufficient for analysis compared to a method not comprising steps c) and/or d).

Exemplary Embodiment 122: The method of embodiment 121, wherein the incidence of tissue insufficient for analysis is reduced by at least 10%, at least 20%, at least 30%, at least 40% or at least 50% compared to the method not comprising steps c and/or d).

Exemplary Embodiment 123: The method of any one of embodiments 1-8 or 10-122, wherein the method step b) comprises extracting the sample using a needle and wherein the method results in a higher tumor purity compared to a method wherein step b) comprises extracting the sample using a razor blade.

Exemplary Embodiment 124: The method of embodiment 123, wherein tumor purity is increased at least at least 10%, at least 20%, at least 30%, at least 40% or at least 50% compared to a method wherein step b) comprises extracting the sample using a razor blade.

Exemplary Embodiment 125: The method of any one of embodiments 20-124, wherein the individual is human.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1. Exemplary Protocol for Precision Enrichment

The following example describes an exemplary protocol for performing precision enrichment of pathology specimens from a formalin-fixed paraffin-embedded (FFPE) block.

First, spatial variation/heterogeneity in the cellular content of a specimen is evaluated to identify target region(s) with representative cells of interest for profiling. An Haematoxylin and Eosin (H&E) slide is analyzed, and one or more 1-3 mm circle(s) are drawn around the region(s) with the greatest density of tumor cells (see FIG. 1).

Next, the target cells are identified in the FFPE block. The H&E slide is aligned with the matched block to create a search image in the block of the target.

Next, a needle is inserted into the target in the block. The needle is a disposable, thin-walled, blunt tipped, stainless steel, hypodermic needle with a Luer-compatible hub. The disposable needle tip is attached to a re-usable handle apparatus such as a syringe. The hypodermic needle bore is typically 14-20 gauge. After punching the block, a post-punch quality control (QC) slide is created from the block. The post-punch QC slide is reviewed in comparison to the pre-punch target slide to inform and document precisely what area of the specimen was punched, and also to inform the cellular content and spatial orientation of the residual specimen block

Next, the needle is pulled out of the block by the handle, and the needle tip is detached from the handle.

Next, a snug-fitting plunger mechanism is used to push the FFPE specimen core out of the needle tip onto the bench for inspection. The plunger may be a paperclip or other piece of wire that fits snuggly inside the needle.

Next, the tissue core is inspected and sliced to remove excess paraffin and further enrich the core as needed.

Finally, the final core(s) are transferred to a digest tube for input into regulated testing chemistry/processes (such as digestion, DnaX treatment, library construction, hybrid capture, etc.).

### Example 2. Increased Tumor Purity and Improved Biomarker Detection Using Precision Needle Punch Enrichment of Pathology Specimen Paraffin Blocks

Background: While many sequencing assays may be geared for short variants (SV), more complex biomarkers such as genomic loss of heterozygosity (gLOH) score, also referred to as homologous recombination deficiency (HRD) score, require higher tumor purity for confident detection. Practical methods to increase tumor nuclei percentage (TN %) from pathology specimens are needed to achieve biomarker results to maximize patient matching to approved therapies and/or clinical trial enrollment.

Methods: Tumor purity of specimens was determined by the computational analysis pipeline component of the FDA-approved NGS assay, FoundationOneCDx. In the validation study, specimen purities for each tissue block were compared following either no enrichment (UnE, n=46), pathologist-directed enrichment by straight razor blade (RBE, n=30) or precision needle punch (NPE, n=47). Post-enrichment Haematoxylin and Eosin (H&E) slides confirmed target region sampled for the NPE arm (see FIG. 2A). Based upon validation data, the needle punch process was implemented for the Lung-MAP prospective clinical trial (LM-NPE). TN % was compared between the first 55 tested LM-NPE specimens and the validation study to assess performance on real-world samples outside of a controlled validation experiment.

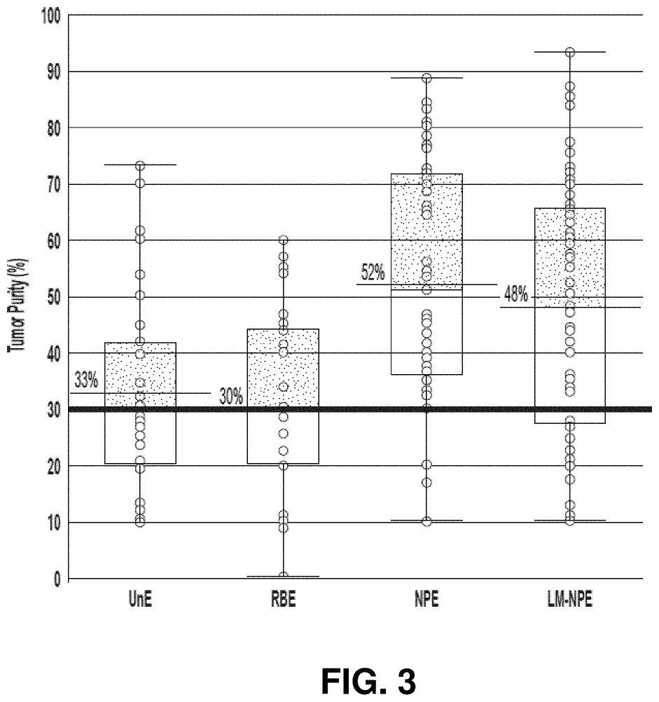

Results: The mean computational TN % in the 4 groups were: UnE: 33%; RBE: 30%; NPE: 52%; and LM-NPE: 48% (FIG. 3). In the validation study, NPE had significantly higher purity than both UnE and RBE (p<0.001); in the trial arm, LM-NPE performed equivalently to NPE (p=0.344). Based upon a 30% tumor purity cutoff, gLOH could be determined for 52% UnE, 50% RBE, 89% NPE and 71% LM-NPE. Comparing NPE and LM-NPE groups reveals no statistical difference in Pass/Fail rates for gLOH determination (p=0.883; Fisher's Test).

Conclusions: Precision needle punch cores from tissue blocks have elevated tumor purity, and consequently, a greater number of successful gLOH determinations. Moreover, this process is rapid and inexpensive. Precision punches may constitute best practice with respect to enriching tumor cells from low-purity specimens for biomarker detection in a routine laboratory specimen-processing setting.

### Example 3. Precision Enrichment Improves Detection of Genomic Loss of Heterozygosity

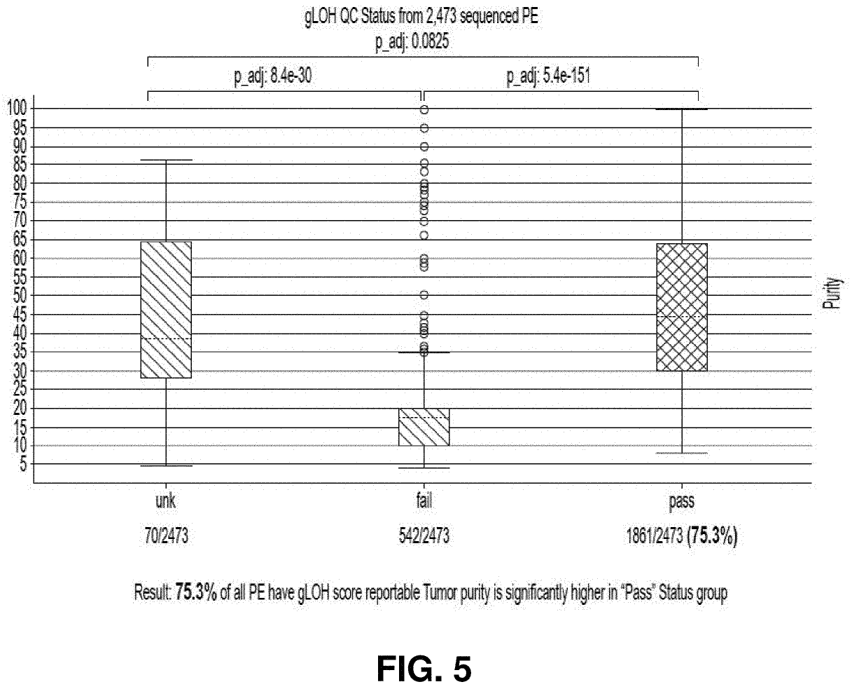

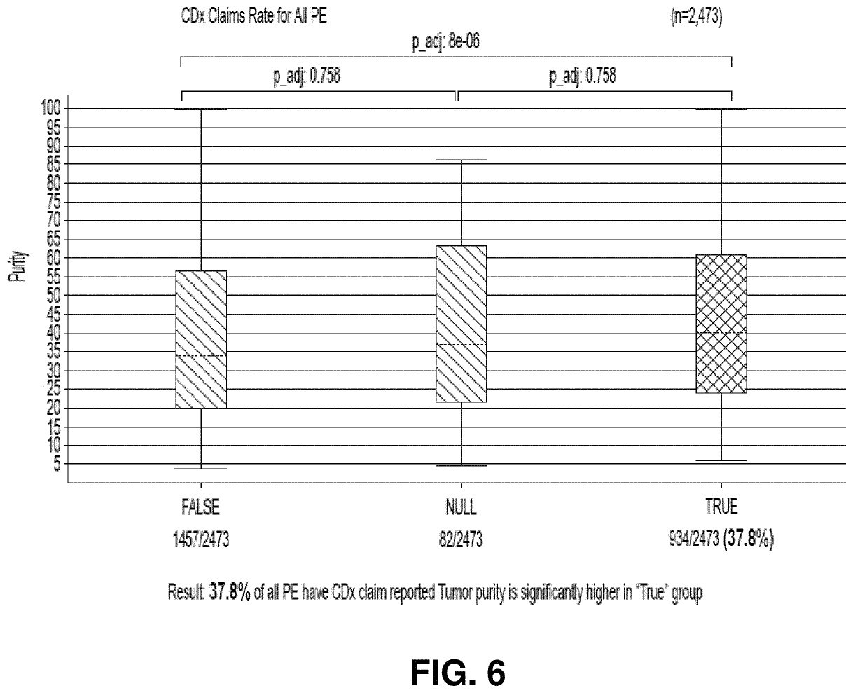

Analyses were performed to test the ability of the precision enrichment method as described in Example 1 to perform comprehensive genome profiling (as diagrammed, e.g., in FIGS. 4A-4B) and detect gLOH, as compared to a method without precision enrichment (i.e., without a step of assessing a post-needle punch quality control slide). As shown in FIGS. 5-6, assessing the post-punch quality control (QC) slide improved detection of gLOH.

### Example 4. Effect of Precision Enrichment on Detection of Loss-of-Function Mutations in a PTEN Gene

An analysis is performed to test the ability of the precision enrichment method as described in Example 1 to perform comprehensive genome profiling and detect loss of function mutations in a PTEN gene to an extract.

### Example 5. Effect of Precision Enrichment on Detection of Tumor Mutational Burden (TMB)

Analyses were performed to test the ability of the precision enrichment method described in Example 1 to perform comprehensive genome profiling (as diagrammed, e.g., in FIGS. 4A-4B) and detect TMB, as compared to a method without precision enrichment. As shown in Table 1 use of precision enrichment improved ability to detect TMB. Specifically, use of the precision enrichment method provided a statistically significant increase in the ability to report elevated TMB (TMB-High) from solid tumor specimens.

The chi-square statistic for this analysis was 56.8966, the p-value was <0.00001, wherein the p-value was considered significant at <0.05. The chi-square statistic with Yates correction for this analysis was 56.7914, the p-value is <0.00001, wherein the p-value was considered significant at <0.05.

TABLE 1
Chi-square statistic analysis of precision enrichment on tumor mutational burden
(TMB)

| | TMB High | TMB not high | Marginal Row Totals |
|---|---|---|---|
| No enrichment | 12078 (12619) [23.19] | 66427 (65886) [4.44] | 78505 |
| Precision enrichment | 12459 (11918) [24.56] | 61685 (62226) [4.7] | 74144 |
| Sample total | 24537 | 128112 | 152649 Grand total) |

### Example 6. Effect of Precision Enrichment on Detection of Homozygous Single Exon Loss

An analysis is performed to test the ability of the precision enrichment method as described in Example 1 to perform comprehensive genome profiling and detect homozygous single exon loss.

### Example 7. Effect of Precision Enrichment on Tissue Insufficient for Analysis (TIFA) Metric

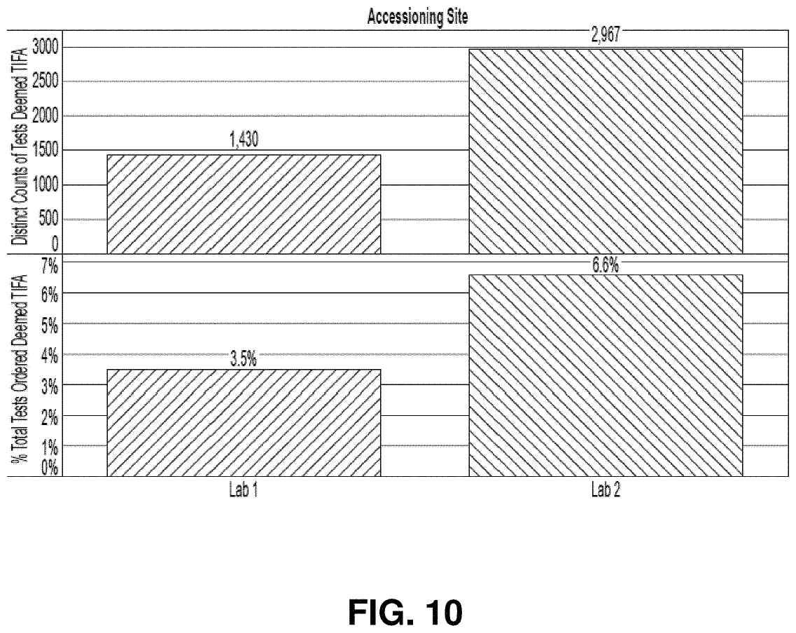

Tissue insufficient for analysis (TIFA) is a measure of how often the tumor tissue content is below 20% for a sample. At lower concentrations, reliable analysis is not possible for certain investigations. An analysis comparing the TIFA rate between two labs was completed (FIG. 10), wherein one lab had access to the precision enrichment protocol from Example 1 (lab 1) and the other lab did not (lab 2). This data demonstrated that availability of the precision enrichment protocol resulted in reduced TIFA levels (i.e., fewer instances of insufficient data for analysis), suggesting more specimens were able to be analyzed (e.g., samples that may have had insufficient tumor tissue content if processed without precision enrichment) and fewer samples failed for lack of sufficient tumor content.

1.-125. (canceled)

126. A method of treating or delaying progression of cancer, comprising:
(I) acquiring knowledge of
loss of heterozygosity (LOH) of one or more genes of interest, a loss-of-function of one or more genes of interest, the level of tumor mutational burden (TMB), or homozygous single exon loss in a sample by
a) identifying a target region comprising tumor cells of interest in a tissue;
b) extracting the sample from the tissue;
c) identifying the location of the sample in the tissue; and
d) if the location of the sample overlaps with the target region comprising tumor cells of interest, extracting one or more nucleic acids from the sample; and
(II) responsive to said knowledge, administering to the individual an effective amount of a treatment that comprises an anti-cancer therapy.

127. A method of improving sequencing analysis, wherein the method comprises:
(I) acquiring knowledge of
loss of heterozygosity (LOH) of one or more genes of interest, a loss-of-function of one or more genes of interest, the level of tumor mutational burden (TMB), or homozygous single exon loss in a sample by
a) identifying a target region comprising tumor cells of interest in a tissue;
b) extracting the sample from the tissue;
c) identifying the location of the sample in the tissue; and
d) if the location of the sample overlaps with the target region comprising tumor cells of interest, extracting one or more nucleic acids from the sample.

128. The method of claim 126, wherein if the location of the sample does not overlap with the target region comprising tumor cells of interest, steps b) and c) are repeated.

129. The method of claim 126, wherein the loss of heterozygosity (LOH) is LOH of a human leukocyte antigen (HLA) gene or the loss-of-function comprises detecting a loss-of-function mutation in a PTEN gene.

130. The method of claim 126, wherein the method further comprises:
ligating one or more adaptors onto one or more nucleic acids extracted from the sample to produce ligated nucleic acids;
amplifying one or more nucleic acids from the ligated nucleic acids to produce amplified nucleic acids;
capturing a plurality of the amplified nucleic acids corresponding to one or more genes of interest to produce captured nucleic acids;
sequencing, by a sequencer, the plurality of the captured nucleic acids to obtain a plurality of sequence reads corresponding to the one or more genes of interest;
analyzing the plurality of sequence reads; and
based on the analysis, detecting one or more mutations in the one or more genes of interest.

131. The method of claim 126, wherein step b) comprises extracting the sample using a needle.

132. The method of claim 131, wherein the needle is punched through the tissue, thereby extracting the sample.

133. The method of claim 132, wherein the needle is a disposable needle.

134. The method of claim 126, wherein the tissue is embedded in an embedding agent.

135. The method of claim 134, wherein the embedding agent is resin or paraffin.

136. The method of claim 126, wherein the one or more nucleic acids extracted from the sample are analyzed by next-generation sequencing.

137. The method of claim 126, wherein the tissue is from a biopsy.

138. The method of claim 126, wherein acquiring knowledge of (i) loss of heterozygosity (LOH) of one or more genes of interest, (ii) a loss-of-function of one or more genes of interest, (iii) the level of tumor mutational burden (TMB), or (iv) homozygous single exon loss in a sample extracted from a tissue from an individual further comprises:
e) providing the tissue comprising tumor cells of interest;
f) extracting a sample from the tissue;
g) assessing the level of enrichment of the tumor cells of interest in the sample and in the remaining tissue; and
h) if the level of enrichment of tumor cells of interest in the sample exceeds the level of tumor cells of interest in the remaining tissue or if the level of enrichment of tumor cells of interest in the sample exceeds a minimum threshold of tumor cells of interest, extracting one or more nucleic acids from the sample.

139. The method of claim 138, wherein the minimum threshold is 25% tumor cells of interest.

140. The method of claim 130, wherein the one or more genes of interest in the sample comprise ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEGS, NNAT, GNAS, and/or GATA5.

141. The method of claim 126, wherein the method reduces the incidence of tissue insufficient for analysis compared to a method not comprising steps c) and/or d).

142. The method of claim 141, wherein the incidence of tissue insufficient for analysis is reduced by at least 10% compared to the method not comprising steps c and/or d).

143. The method of claim 126, wherein the method step b) comprises extracting the sample using a needle and wherein the method results in a higher tumor purity compared to a method wherein step b) comprises extracting the sample using a razor blade.

144. The method of claim 143, wherein tumor purity is increased at least at least 10% compared to a method wherein step b) comprises extracting the sample using a razor blade.

145. The method of claim 126, wherein the individual is human.

146. A method of treating or delaying progression of cancer, comprising, responsive to acquiring knowledge of (i) loss of heterozygosity (LOH) of one or more genes of interest, (ii) a loss-of-function of one or more genes of interest, (iii) the level of tumor mutational burden (TMB), or (iv) homozygous single exon loss in a sample extracted from a tissue from an individual according to the method of claim 126, administering to the individual an effective amount of a treatment that comprises an anti-cancer therapy.

147. A method of identifying an individual having cancer who may benefit from a treatment comprising an anti-cancer therapy, the method comprising detecting (i) loss of heterozygosity (LOH) of one or more genes of interest, (ii) a loss-of-function of one or more genes of interest, (iii) the level of tumor mutational burden (TMB), or (iv) homozygous single exon loss in a sample extracted from a tissue from the individual according to the method of claim 126, wherein (i) the presence of LOH of one or more genes of interest, (ii) the presence of the loss-of-function of one or more genes of interest, (iii) the level of TMB, or (iv) the presence of homozygous single exon loss in the sample identifies the individual as one who may or may not benefit from the anti-cancer therapy.

148. The method of claim 147, wherein the loss of heterozygosity (LOH) is LOH of a human leukocyte antigen (HLA) gene or the loss-of-function comprises detecting a loss-of-function mutation in a PTEN gene.

149. The method of claim 147, wherein the one or more genes of interest in the sample comprise ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEGS, NNAT, GNAS, and/or GATA5.

150. A method of detecting the presence or absence of a cancer in an individual comprising detecting (i) the presence or absence of loss of heterozygosity (LOH) of one or more genes of interest, (ii) the presence or absence of a loss-of-function of one or more genes of interest, (iii) the level of tumor mutational burden (TMB), or (iv) the presence or absence of homozygous single exon loss in a sample extracted from a tissue from the individual according to the method of claim 126.

151. A method of selecting a therapy for an individual having cancer comprising detecting (i) loss of heterozygosity (LOH) of one or more genes of interest, (ii) a loss-of-function of one or more genes of interest, (iii) the level of tumor mutational burden (TMB), or (iv) homozygous single exon loss in a sample extracted from a tissue from the individual according to the method of claim 126, wherein (i) the presence of LOH of one or more genes of interest, (ii) the presence of the loss-of-function of one or more genes of interest, (iii) the level of TMB, or (iv) the presence of homozygous single exon loss in the sample individual identifies the individual as one who may benefit from a treatment comprising an anti-cancer therapy.

152. A method of selecting or not selecting a treatment for an individual having cancer, comprising acquiring knowledge of (i) loss of heterozygosity (LOH) of one or more genes of interest, (ii) a loss-of-function of one or more genes of interest, (iii) the level of tumor mutational burden (TMB), or (iv) homozygous single exon loss in a sample extracted from a tissue from the individual having cancer according to the method of claim 126, wherein responsive to the acquisition of said knowledge: (i) the individual is classified as a candidate to receive treatment with an anti-cancer therapy, or the individual is not classified as a candidate to receive treatment with an anti-cancer therapy; and/or (ii) the individual is identified as likely to respond to a treatment that comprises an anti-cancer therapy, or the individual is identified as unlikely to respond to a treatment that comprises an anti-cancer therapy.

153. A method of assessing (i) loss of heterozygosity (LOH) of one or more genes of interest, (ii) a loss-of-function of one or more genes of interest, (iii) the level of tumor mutational burden (TMB), or (iv) homozygous single exon loss in a cancer in an individual, the method comprising:
(a) detecting (i) LOH of one or more genes of interest, (ii) the loss-of-function of one or more genes of interest, (iii) the level of TMB, or (iv) homozygous single exon loss in a sample extracted from a tissue from the individual according to the method of claim 126; and
(b) providing an assessment of (i) the LOH of one or more genes of interest, (ii) the loss-of-function of one or more genes of interest, (iii) the level of TMB, or (iv) the homozygous single exon loss.

154. A method of detecting the presence or absence of a cancer in an individual, the method comprising detecting (i) the presence or absence of loss of heterozygosity (LOH) of one or more genes of interest, (ii) the presence or absence of LOH of an HLA gene, (iii) the presence or absence of a loss-of-function mutation in a PTEN gene, (iv) the level of tumor mutational burden (TMB), or (v) the presence or absence of homozygous single exon loss in a sample extracted from a tissue from the individual according to the method of claim 126.

155. A system comprising:
a memory configured to store one or more program instructions; and
one or more processors configured to execute the one or more program instructions, wherein the one or more program instructions when executed by the one or more processors are configured to:
(a) obtain a plurality of sequence reads of one or more nucleic acids, wherein the one or more nucleic acids are derived from a sample extracted from a tissue according to claim 126;
(b) analyze the plurality of sequence reads for the presence of loss of heterozygosity (LOH) of one or more genes of interest, LOH of a human leukocyte antigen (HLA) gene, a loss-of-function mutation in a PTEN gene, tumor mutational burden (TMB), and/or homozygous single exon loss; and
(c) detecting cancer that may be treated with a treatment that comprises an anti-cancer therapy, based on the analyzing, LOH of one or more genes of interest, LOH of a HLA gene, a loss-of-function mutation in a PTEN gene, TMB, and/or homozygous single exon loss in the sample.
